(19) United States
(12) Patent Application Publication
(10) Pub. No.: US 2025/0195496 A1
(43) Pub. Date: Jun. 19, 2025

(54) AVPR1A BLOCKADE TO REDUCE SOCIAL ISOLATION-INDUCED ANXIETY IN FEMALES

(71) Applicant: THE TRUSTEES OF COLUMBIA UNIVERSITY IN THE CITY OF NEW YORK, New York, NY (US)

(72) Inventors: Lori Michelle ZELTSER, New York, NY (US); Marie Louise Pascale Jocelyna FRANCOIS, New York, NY (US)

(21) Appl. No.: 18/814,287

(22) Filed: Aug. 23, 2024

Related U.S. Application Data
(63) Continuation of application No. PCT/US2023/063377, filed on Feb. 27, 2023.
(60) Provisional application No. 63/485,248, filed on Feb. 15, 2023.
(60) Provisional application No. 63/314,690, filed on Feb. 28, 2022.

(51) Int. Cl. A61K 31/4545; A61P 25/22

(52) U.S. Cl. CPC A61K 31/4545; A61P 25/22

(57) ABSTRACT

Treatment of females that experience social isolation-induced anxiety includes administration of an agent that blocks or reduces AVPR1A signaling in the subject. Such agents may interfere with AVPR1A in the amygdala, or specifically in the central nucleus of the amygdala (CeA). Treatment may be administered by injection or by chemogenetic approaches. Compounds that block AVPR1A signaling may be used to accomplish such treatment. Useful compounds include, for example, AVPR1A antagonists such as SRX246.

### CROSS-REFERENCE TO RELATED APPLICATIONS

This application is a continuation of International Patent Application No. PCT/US2023/063377, filed Feb. 27, 2023, which claims priority to U.S. Provisional Application No. 63/314,690 filed on Feb. 28, 2022, and U.S. Provisional Application No. 63/485,248 filed on Feb. 15, 2023, the entire contents of which applications are incorporated herein by reference thereto.

### GOVERNMENT LICENSE RIGHTS

This invention was made with government support under grant no. MH113353 awarded by the National Institutes of Health (NIH). The government has certain rights in the invention.

### TECHNICAL FIELD

The disclosure of the present patent application relates to new therapeutic methods for treating social isolation-induced anxiety in females, comprising administration of arginine vasopressin receptor 1a (AVPR1A) antagonists. Females are more sensitive to social exclusion, which could contribute to their heightened susceptibility to anxiety disorders. Chronic social isolation stress (CSIS) for at least 7 weeks after puberty induces anxiety-related behavioral adaptations in female mice.

### DESCRIPTION OF THE RELATED ART

Anxiety disorders are the second-most common mental health disorder, with a higher lifetime prevalence in women according to epidemiological surveys (Baxter et al., 2013; Collaborators, 2021; Kessler et al., 2005; Kessler et al., 2012; Pine et al., 1998; Wittchen et al., 1998). The incidence increases dramatically after puberty and declines in parallel with the reproductive period of females (Collaborators, 2021; Craske, 2003; Kessler et al., 2012; Pine et al., 1998; Wittchen et al., 1998). Sex differences in susceptibility to anxiety disorders are magnified across adolescence to young adulthood, reaching ratios of 2:1 to 3:1 (Craske, 2003; Pine et al., 1998; Wittchen et al., 1998). However, the underlying neurobiological mechanisms driving these sex differences are unknown.

Exposure to chronic stress, and to social stress in particular, has been implicated in the etiology of anxiety disorders (Brown, 1993; McEwen and Stellar, 1993; Patriquin and Mathew, 2017). Sex differences in responsiveness to distinct types of social stressors complicate efforts to explore their contributions to the pathophysiology of anxiety disorders in clinical studies. Men react more to achievement or ego-threatening stress, while women respond more to social exclusion stress (Benenson et al., 2013; Clauss and Byrd-Craven, 2019; Stroud et al., 2002). In a meta-analysis of neuroimaging studies, females exhibit more robust neural responses to negative emotions, while males are more responsive to positive emotions; this valence-specificity was most robust in the amygdala (Stevens and Hamann, 2012). Sex differences in stress responses could partly explain increased susceptibility of females to anxiety disorders (Ordaz and Luna, 2012; Rutter et al., 2003; Stevens and Hamann, 2012).

Age- and sex-specific responses to different types of stress have also been observed in pre-clinical rodent models (Beck and Luine, 2002; Donner and Lowry, 2013; Goel and Bale, 2009; Tan et al., 2021). These likely reflect differences in brain circuits regulating and responding to changing social relationships across development. During the juvenile period (P21-35), pups engage in playful interactions with cage mates that are critical to the maturation of social behaviors (Arakawa, 2018). Social isolation during this period disrupts the establishment of these behaviors, with lasting effects on behavioral responses to stress (Walker et al., 2019). After puberty, males develop territorial and dominant-subordinate relationships (Luciano and Lore, 1975); elimination of these interactions therefore does not promote anxiety-related behavioral adaptations (Hilakivi et al., 1989; Liu et al., 2013; Rivera-Irizarry et al., 2020; Yorgason et al., 2013; Zelikowsky et al., 2018). In contrast, females maintain positive social relationships with siblings into adulthood, and groups of females typically live together in communal nests (Manning et al., 1995). Social isolation after puberty deprives female rodents of these desired relationships, and thus induces behavioral disturbances that are thought to model anxiety (Palanza, 2001; Rivera-Irizarry et al., 2020). Thus, there is a growing appreciation for the need to develop sex-specific assays to study stress (Francois et al., 2022; Furman et al., 2022; Haller et al., 1999; Palanza, 2001; Takahashi et al., 2017).

### SUMMARY

Anxiety disorders, the second-most common mental health disorder, are more prevalent in females. Exposure to chronic social stress has been implicated in the etiology of anxiety disorders, and females are more susceptible to social exclusion and loneliness, raising the possibility that they are mechanistically linked. Here we used a mouse model of chronic social isolation stress (CSIS) to uncover a neural circuit (AVP^{MeA}→AVPR1A^{CeA}→CPu) and molecular pathway (AVPR1A) that mediate sex-specific increases in anxiety-related behaviors.

Key findings include:
Chronic (>7 weeks) social isolation in females, but not males, increases anxiety-related behaviors.
Avpr1a expression in the central nucleus of the amygdala (CeA) is upregulated in females, and not males, exposed to CSIS; this persists even after mice are re-grouped. The long duration of social isolation is a critical feature of this model. Avpr1a expression in the CeA is not affected by other stressors, such as overcrowding or repeated restraint.
Targeted loss of Avpr1a in the CeA and peripheral delivery of AVPR1A antagonists reverse effects of CSIS on anxiety-related behaviors in females but have no effect in males or in group housed females.
The top projection targets of AVPR1A^{CeA} neurons reside in the medial amygdala, caudate putamen (CPu) and midbrain reticular system. This pattern is distinct from the rest of the CeA, which primarily sends descending projections to the brainstem. We further show that AVPR1A^{CeA}→CPu circuits mediate, in part, the effect of CSIS to increase anxiety-related behaviors in females.
Contrary to the current dogma, we show that the posterodorsal medial amygdala (MePD), and not the paraventricular nucleus of the hypothalamus, is a major source of AVP projections to the CeA. Knockdown of Avp in the MePD or loss of estrogen receptor α in AVP^{MePD} neurons diminishes the effect of CSIS on anxiety-related behaviors in females.

Many groups have utilized functional circuit mapping approaches to modulate anxiety-related behavior in mice. The significance of this manuscript lies in the identification of a system that is endogenously activated in the context of chronic social isolation exclusively in females. This discovery is timely, as social restrictions imposed during the COVID-19 pandemic were associated with an estimated 76.2 million additional cases of anxiety disorders across the globe, particularly in young adult females. Our findings demonstrate that AVPR1A antagonists that are currently in clinical trials (which are mostly targeting aggressive behaviors in males) could be effective in females experiencing social rejection and/or loneliness. These studies are consistent with the growing call to develop therapeutics that target the etiology of a psychiatric disorder (i.e., social isolation), rather than its symptoms.

Transitory blockade of the central amygdala AVPR1A pathway with chemogenetic inhibition of these neurons is sufficient to abolish anxiety and OCD-like behaviors in socially isolated female mice, and increases dark phase food intake. Moreover, conditional deletion of the AVPR1A gene on these neurons is also sufficient to block these behaviors and ameliorates food intake in females only, showing that these anxiolytic effects are specific to the AVPR1A pathway. Peripheral injection of an AVPR1A antagonist crossing the blood brain barrier (Manning compound, 7 g/kg) is sufficient to decrease OCD-like behaviors in the marble burying assay in females, and to block the anorexigenic effects of AVP (0.3 ng) when injected in the amygdala. These effects are specific to central AVPR1A circuits because peripheral injection of an antagonist that does not cross the blood brain barrier (SR49059, 2 mg/kg) does not restore food intake following amygdala injection of AVP (0.3 ng).

Preliminary results with the use of certain anti-sense oligonucleotides targeting central AVPR1A circuits (ICV, 500 g) to reduce anxiety-like behaviors are promising. These results advance the field in two ways. First, they provide evidence that AVPR1A is a therapeutic target for treat anxiety and OCD in women experiencing social isolation. Second, they show that this pathway is not engaged in socially isolated males. Preclinical data supporting the use of AVPR1A antagonists to treat anxiety or aggression were only generated in males. Moreover, the studies in humans have focused on irritability and aggression in the context of Huntington's Disease, Intermittent Explosive Disorder, and PTSD or social deficits in autism spectrum disorder exclusively in males. This likely stems from the dogma that AVP is the predominant “social” neuropeptide in males, while oxytocin predominates in females.

The AVP system modulates the activity of the neuroendocrine stress axis (Gillies et al., 1982; Griebel et al., 2005), and it is known to contribute to the pathophysiology of emotional and social disorders that have sex-biases (Heinrichs and Domes, 2008; Landgraf, 2006; Meyer-Lindenberg et al., 2011; Neumann and Landgraf, 2012), but its role in the amygdala is less studied. Here we demonstrate that signaling through the AVPR1A pathway is necessary to elicit anxiety-related behavioral responses to CSIS.

We identified a major source of AVP ligand in the posterodorsal part of the medial amygdala (MePD) as well as an important downstream target of AVPR1A^{CeA} neurons, the caudate putamen (CPu). Sex specificity of these effects is mediated, in part, by signaling via estrogen receptor α (ERα) in AVP^{MePD} neurons. These results fill a critical gap in understanding of the neural substrates underlying sex-specificity in vulnerability to CSIS.

### BRIEF DESCRIPTION OF FIGURES

FIG. 1A-FIG. 1L: AVPR1A in the central amygdala mirrors the period of susceptibility to anxiety disorders and activation of AVPR1A^{CeA} neurons elicits anxiety-related behaviors.

(A) Heat map for the expression of the 300 top genes at κ, 7, 13 and 22 weeks of age (n=3). (B) Classification of genes upregulated at 7 weeks of age with Gene Ontology terms based on molecular function. (C) STRING analysis for the genes upregulated at 7 weeks of age within the molecular transducer activity family. (D) Vgat and AVPR1A expression detected with smFISH in a coronal section of the CeA at bregma-1.34 mm. (E) Quantification of AVPR1A^{CeA} neurons. (F) AVPR1A-Cre::tdTomato reporter expression in a coronal section of the CeA at bregma-1.34 mm. (G-L) Chemogenetic activation of AVPR1A^{CeA} neurons. (G) Schematic of bilateral injections of AAV-DIO-DREADD-Gq-mCherry (closed circles) vs. AAV-DIO-mCherry controls (open circles) in the CeA of Avpr1a-Cre adult mice. (H) Expression of the viral mCherry reporter in the CeA. High-magnification image showing Avpr1a and cFos expression detected with smFISH 1 h after CNO injections in mice injected with control (lower panel) and DREADD-Gq-mCherry (upper panel) AAVs. (I-L) Effects of CNO injections on marble burying (I), time spent in the open arms of the EPM (J), time spent in the center of the open field (K), and distance traveled in the open field (L) in mice injected with control and DREADD-Gq AAVs (n=6-9). Data are presented as means+/−SEM in E.

FIG. 2A-FIG. 2L: Avpr1a in the CeA mediates the effects of CSIS on anxiety-related behaviors in adult females.

(A-F) Effects of housing density in WT mice. (A) Quantification of Avpr1a mRNA by qPCR in the CeA of mice exposed to CSIS (n=13-15). (B-C) Effect of CSIS on marble burying (B), time spent in the open arms of the EPM (C). (D) Expression of Avpr1a in the CeA of females that were group housed or socially isolated for 7 weeks (starting at 8 weeks of age) or 2 weeks (starting at 10 weeks of age). (E) Quantification of Avpr1a mRNA by qPCR in the CeA of females that were group housed or socially isolated for 7 weeks (starting at 8 weeks of age) and regrouped for 3 weeks (n=8-10). (F) Effect of 3 weeks of regrouping after CSIS on marble burying in females (n=6). (G-L) Effects of housing density in mice with a targeted deletion of Avpr1a in the CeA. (G) Schematic of bilateral injections of AAV-Cre-GFP (closed circles) vs. AAV-GFP controls (open circles) in the CeA of Avpr1a^{Flox/Flox} mice. (H) Validation of Avpr1a deletion by qPCR in the CeA of Avpr1a^{Flox/Flox} mice injected with AAV-Cre-GFP or AAV-EGFP (controls) (n=5-6). (I-J) Effect of CeA Avpr1a deletion on marble burying behavior (I) and time spent in the open arms of the EPM (J) of Avpr1a^{Flox/Flox} homozygotes. (K-L) Effect of CeA Avpr1a deletion on marble burying behavior (K) and time spent in the open arms of the EPM (L) of Avpr1a^{Flox/+} heterozygotes.

FIG. 3A-FIG. 3H: Blockade of central AVPR1A signals decreases CSIS-induced anxiety-related behavioral adaptations in adult females.

(A-D) Effects of i.p. (Intraperitoneal) injections of AVPR1A antagonists on complex behaviors in adult WT mice that were exposed to >7 weeks of CSIS starting at 5 weeks of age. (A) Effects of SRX246 and SR49059 on marble burying (n=10-12). (B-D) Effect of SRX246 (closed circles) vs. vehicle (open circles) on time spent in the open arms of the EPM (B), time spent in the center of the open field (C), and distance traveled in the open field (D) (n=10-13). (E-H) Effects of i.p. injections of AVPR1A antagonists on complex behaviors in adult WT mice that were exposed to >7 weeks of CSIS starting at 8 weeks of age. (E) Effects of SRX246 and SR49059 on marble burying (n=13-15). (F-H) Effect of SRX246 on time spent in the open arms of the EPM (F), time spent in the center of the open field (G), and distance traveled in the open field (H) (n=10-15).

FIG. 4A-FIG. 4M: AVPR1A^{CeA}→CPu circuits mediate some of the behavioral adaptations to CSIS in females.

(A-E) Anterograde tracing from AVPR1A^{CeA} neurons. (A) Schematic of unilateral AAV-DIO-Synaptophysin-mCherry injections in the CeA of Avpr1a-Cre mice. (B) Expression of the viral mCherry reporter in coronal sections of the CeA. (C) Heat map of AVPR1A^{CeA} neuronal projections throughout the brain (n=3 per sex). (D-E) Expression of the viral mCherry reporter in coronal sections of the CPu in females (D) and males (E). (F-H) Retrograde tracing from the CPu to AVPR1A^{CeA} neurons. (F) Schematic of unilateral AAV-DIO-EYFP injections in the CPu of Avpr1a-Cre mice. (G-H) Expression of the viral EYFP reporter in coronal sections of the CPu (G) and CeA. (I-M) Effects of chemogenetic inhibition of AVPR1A^{CeA}→CPu circuits. (I) Schematic of dual bilateral injections of retrograde AAV-DIO-Flp in the CPu (green) and AAV-fDIO-DREADD-Gi in the CeA (blue) of Avpr1a-Cre mice exposed to CSIS. (J-M) Effects of CNO injections on marble burying (J), time spent in the open arms of the EPM (K), time spent in the center of the open field (L), and distance traveled in the open field (M) (n=4-9).

FIG. 5A-FIG. 5R: The MePD releases AVP to the CeA to increases anxiety-related behaviors during CSIS in females.

(A-B) Retrograde tracing from the CeA to AVP^{MePD} neurons. (A) Schematic of unilateral retrograde AAV-fDIO-mCherry injections in the CeA of Avp-Flp::GFP mice. (B) Co-expression of the viral mCherry reporter in GFP-labeled AVP neurons in coronal sections of the MePD. (C) Avp and Avpr1a mRNA detected with smFISH in coronal sections of the MePD and CeA, respectively. (D-E) Anterograde tracing from AVP^{MePD} neurons to the medial CeA. (D) Schematic of unilateral dual injections of AAV-fDIO-Cre and AAV-DIO-Synaptophysin in the MePD of Avp-Flp::GFP mice. (E) Expression of the GFP in AVP^{MePD} neurons and the viral mCherry reporter in projections to the CeA. (F) Expression of the GFP lineage trace in Avp-expressing cell bodies in the MePD and projections into the CeA relative to the position of AVPR1A^{CeA} neurons marked with a TOM linage trace in Avpr1a-Cre::tdTomato::Avp-Flp::GFP mice. (G-L) CRISPR-mediated knockdown of Avp in the MePD of WT mice exposed to CSIS. (G) Schematic of bilateral injections of a mix of AAV-SaCas9 and AAV-gRNA-AVP-EGFP (closed circles) or AAV-gRNA-Scramble-EGFP (open circles) in the MePD. (H) Validation of Avp knockdown in the MePD of mice injected with AAV-SaCas9 and AAV-gRNA-AVP-EGFP vs. AAV-gRNA-Scramble-EGFP controls (n=4). (I-L) Effects of MePD Avp knockdown on marble burying (I), time spent in the open arms of the EPM (J), time spent in the center of the open field (K), and distance traveled in the open field (L) (n=9-16). (M-R) Loss of Esr1 in AVP^{MePD} neurons in mice exposed to CSIS. (M) Schematic of bilateral injections of AAV-fDIO-Cre (closed circles) vs. AAV-fDIO-mCherry (open circles) in the MePD of Esr1^{Flox/Flox}::Avp-Flp mice. (N) Avp (red) and Esr1 (green) mRNA detected by smFISH in the MePD of mice injected with AAV-fDIO-mCherry (left) or AAV-fDIO-Cre-mCherry (right). (O-R) Effect of Esr1 deletion from Avp-expressing neurons in the MePD on marble burying (O), time spent in the open arms of the EPM (P), time spent in the center of the open field (Q), and distance traveled in the open field (R) (n=5-9).

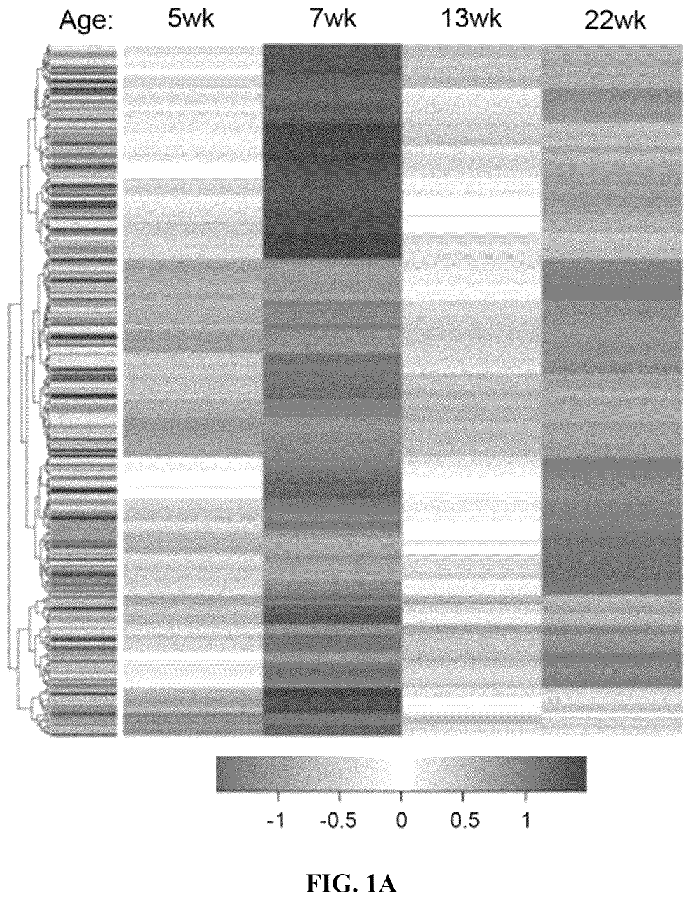

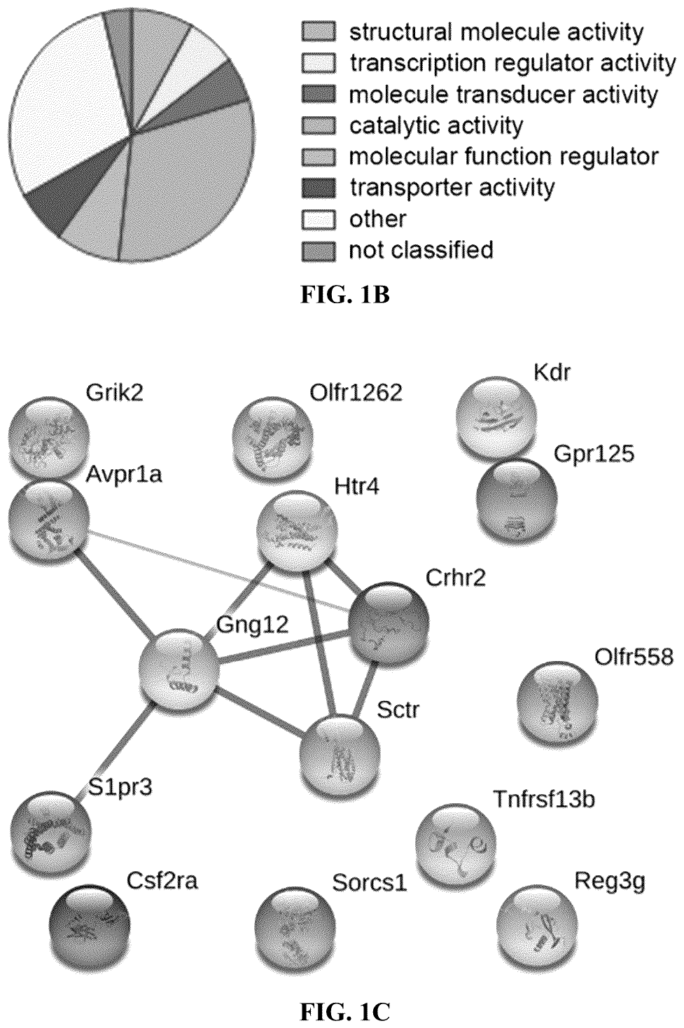

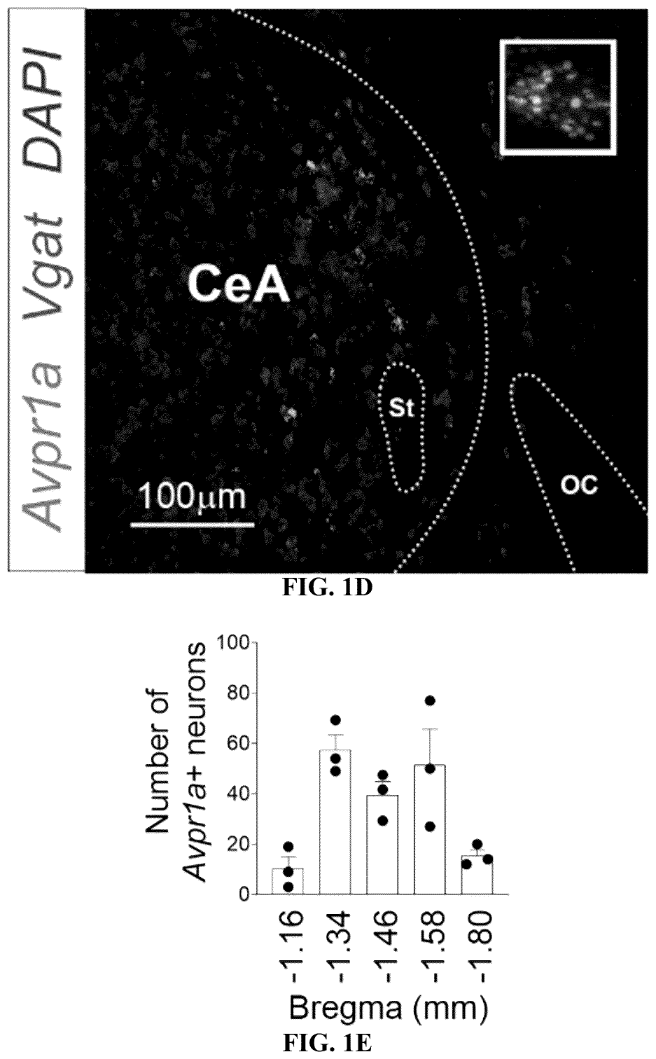

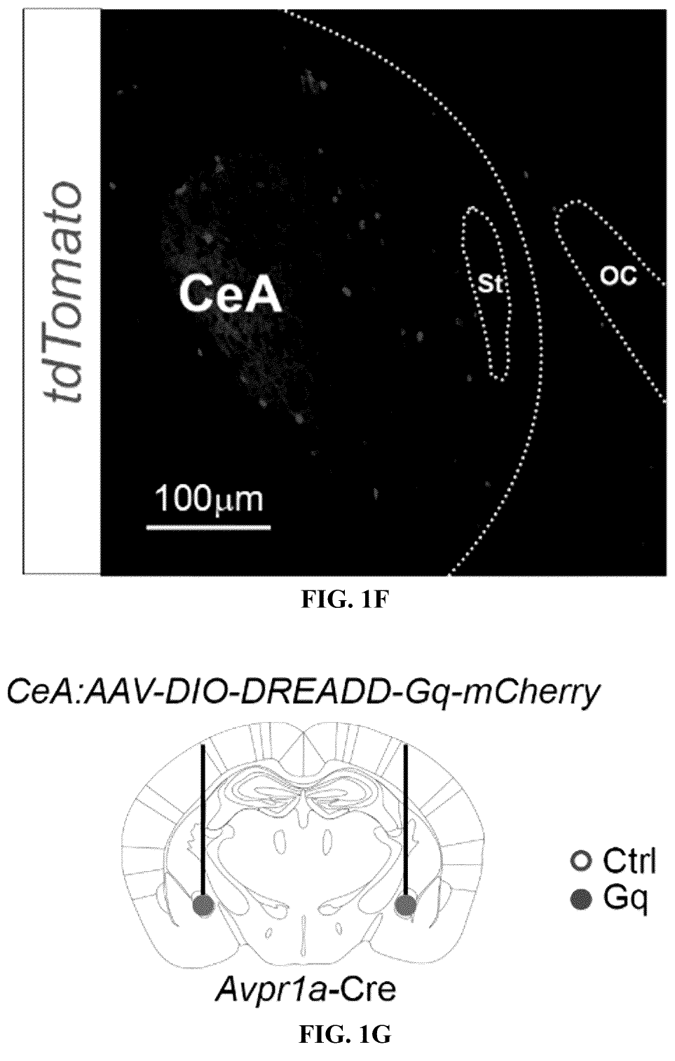

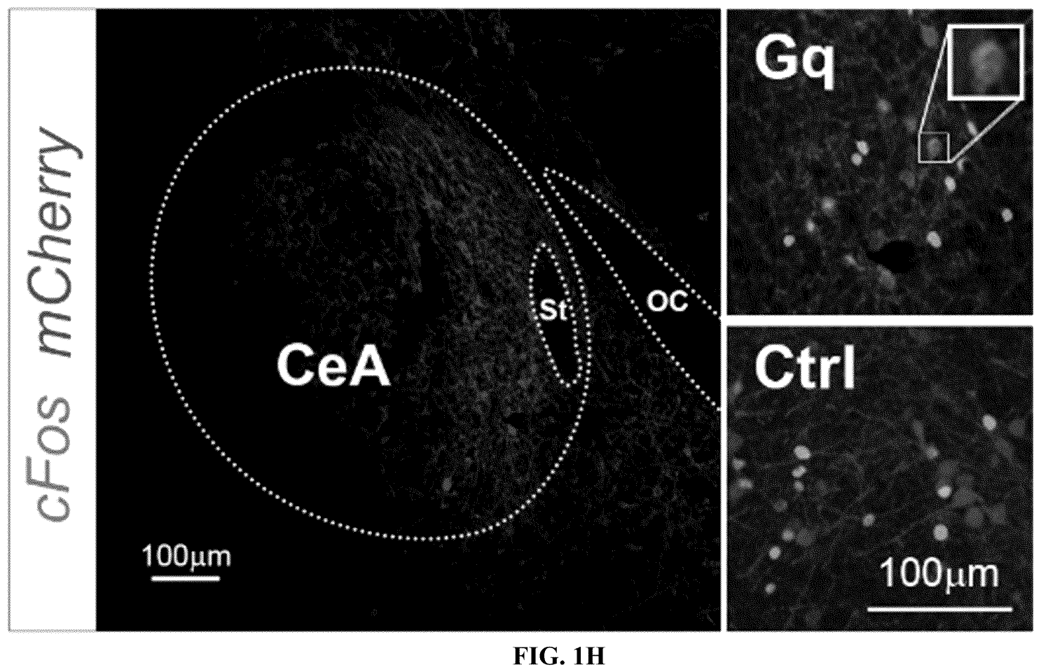

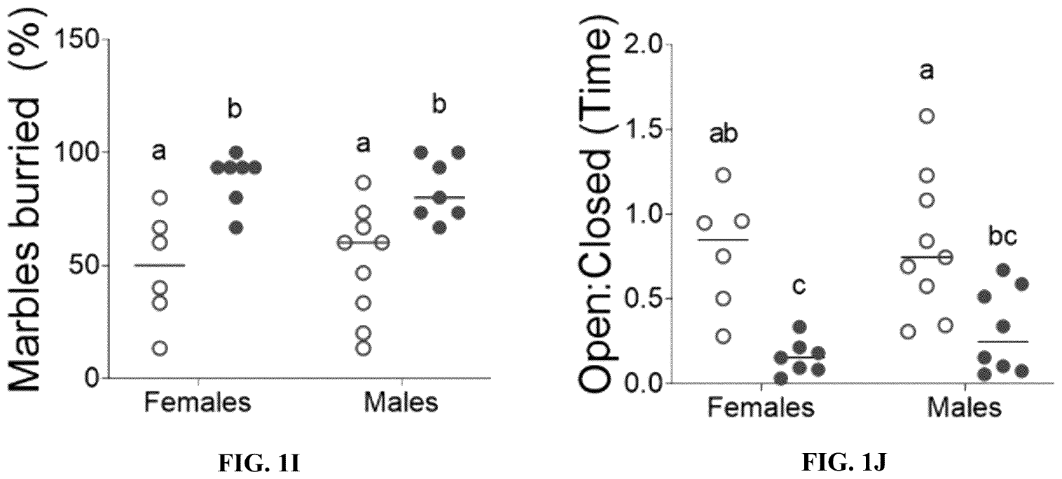

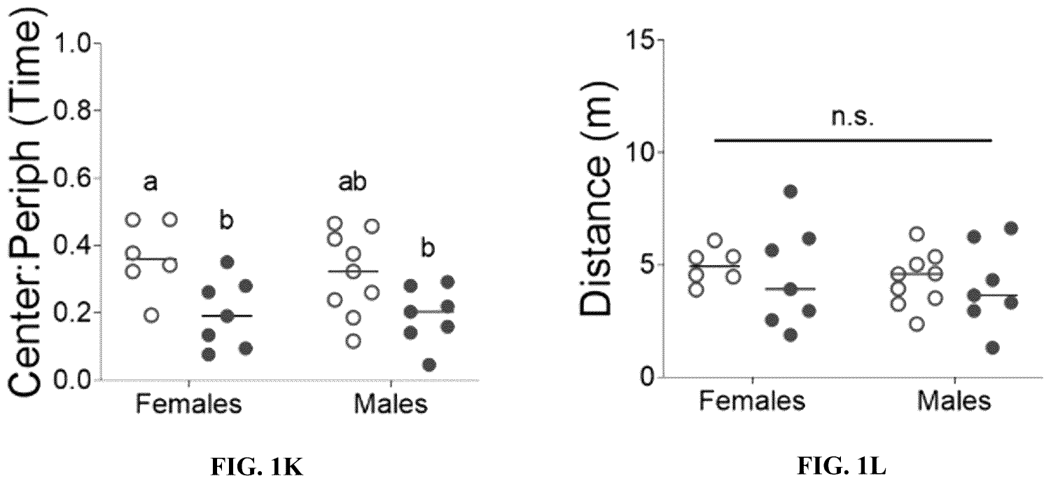

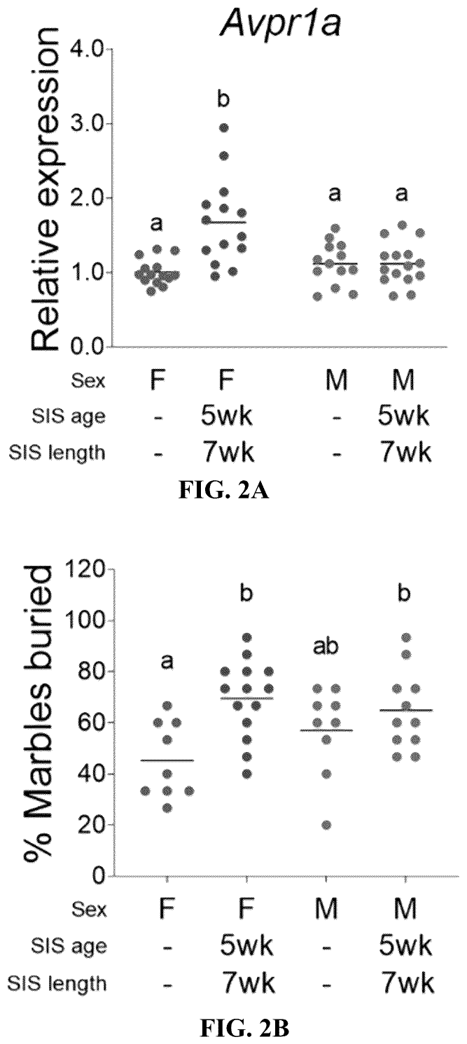

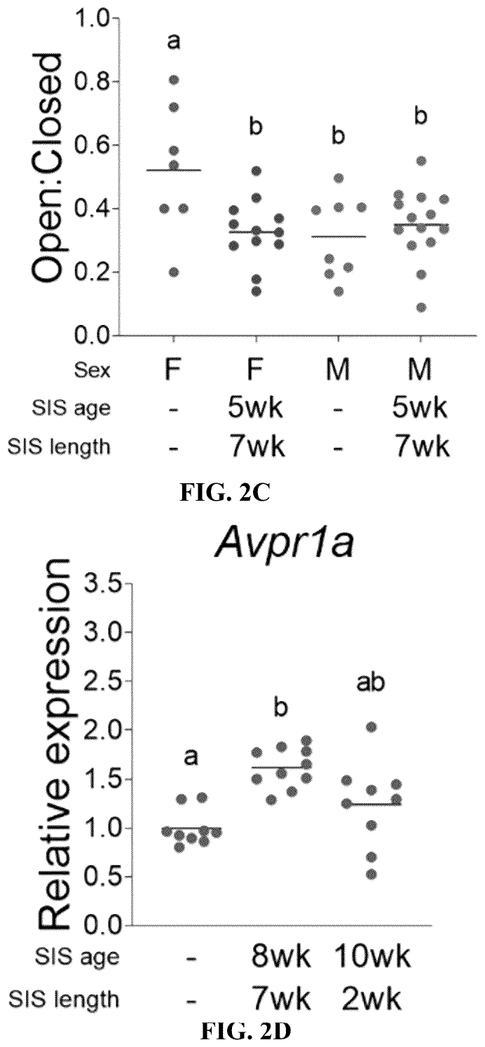

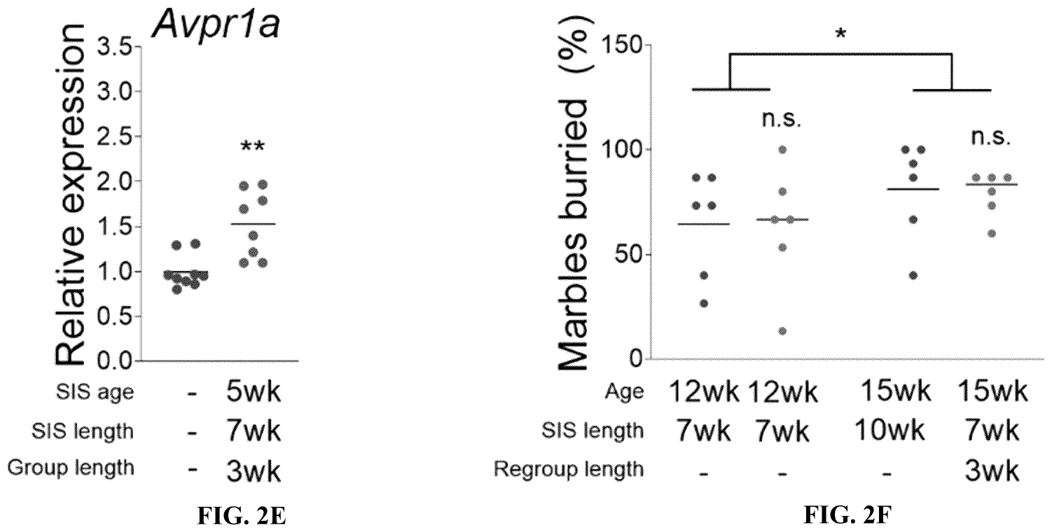

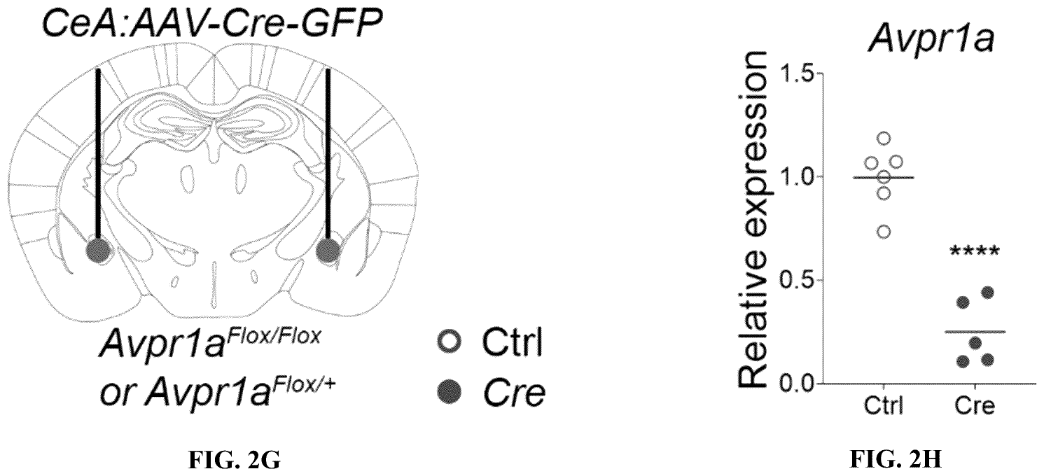

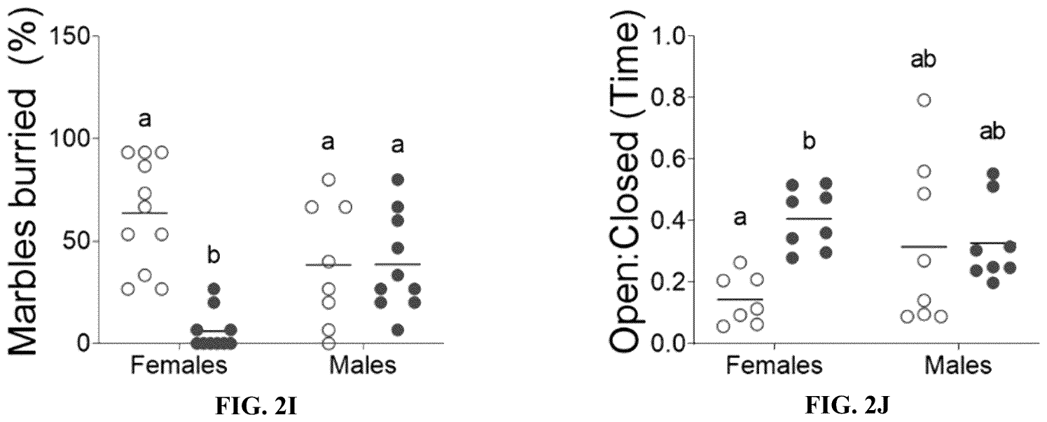

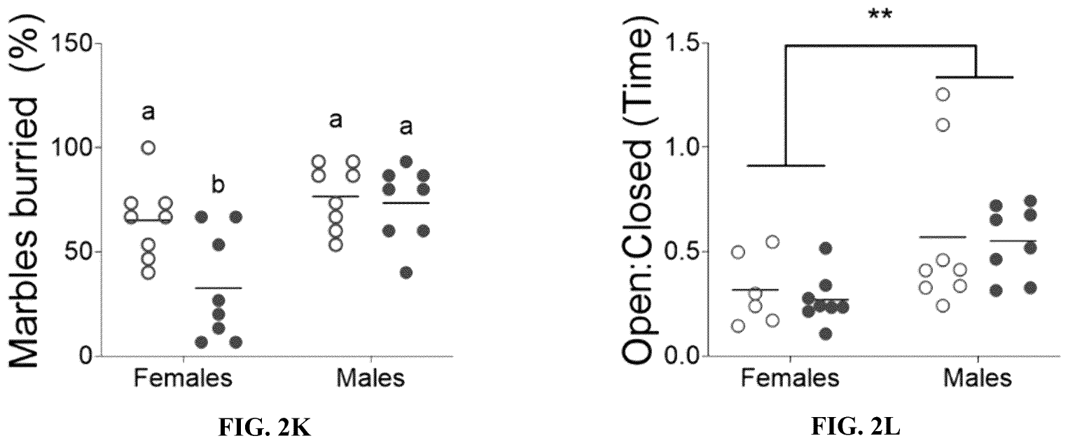

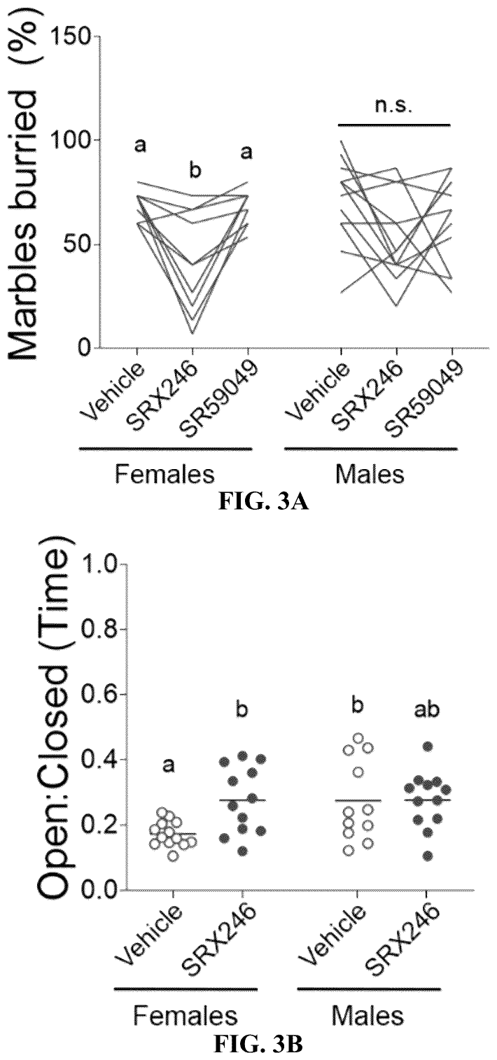

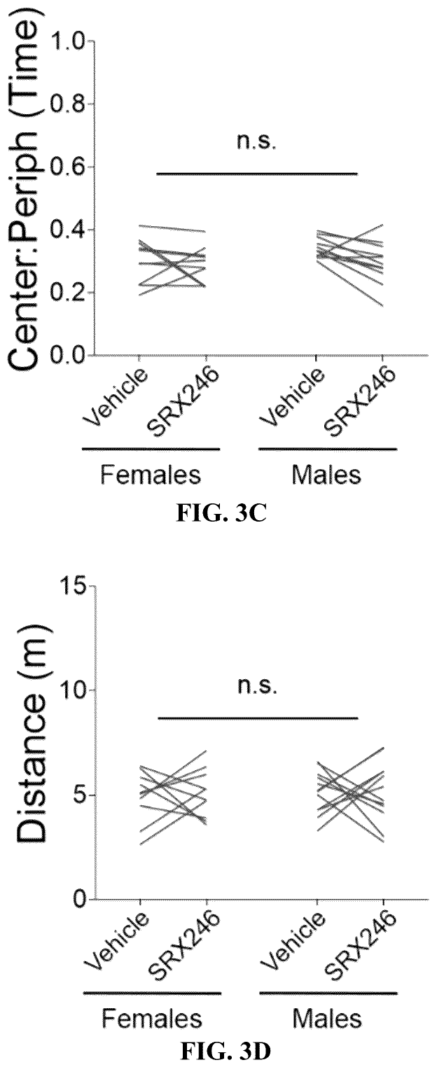

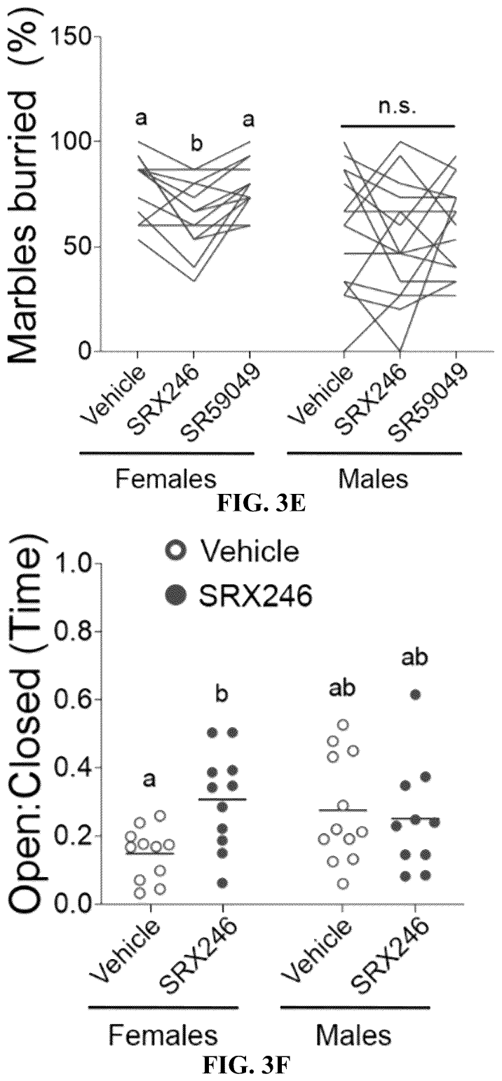

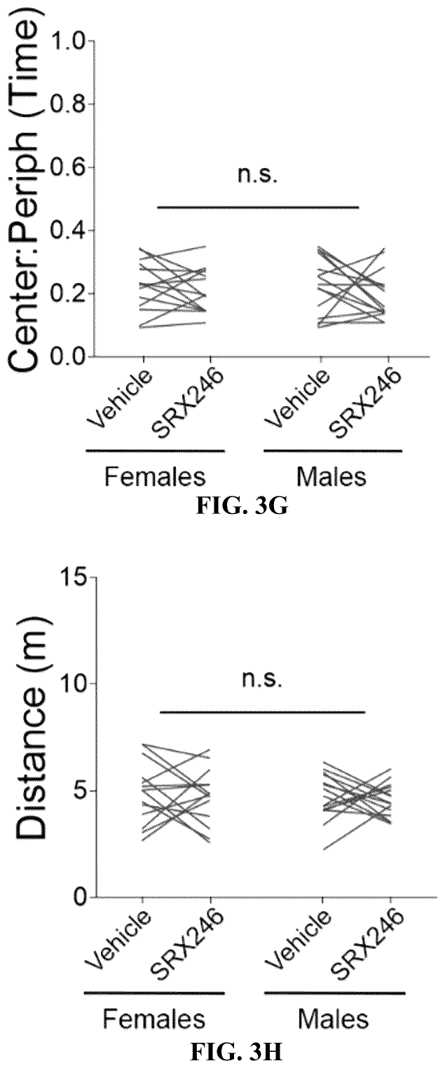

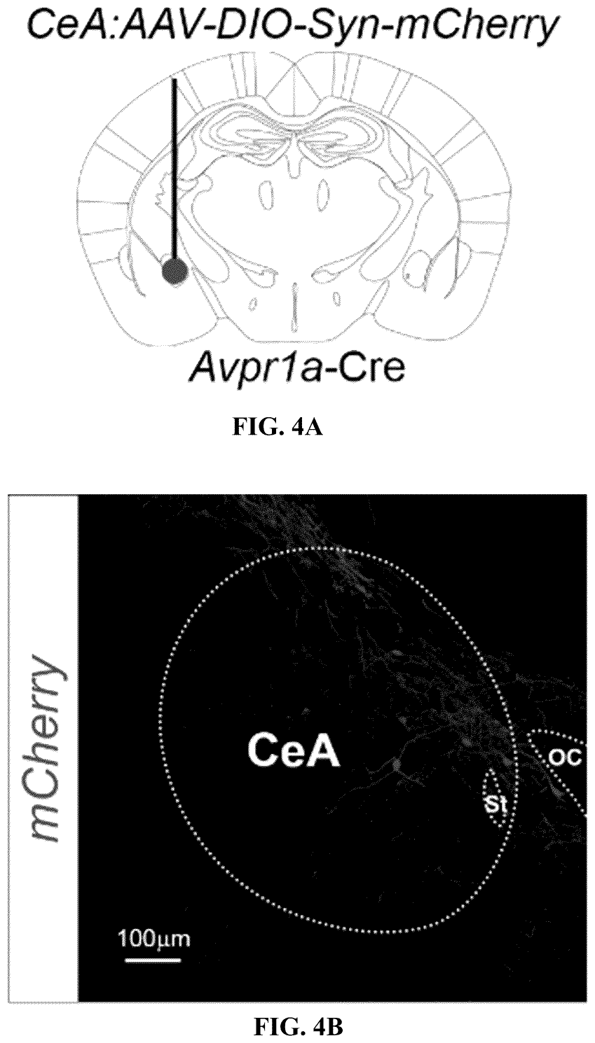

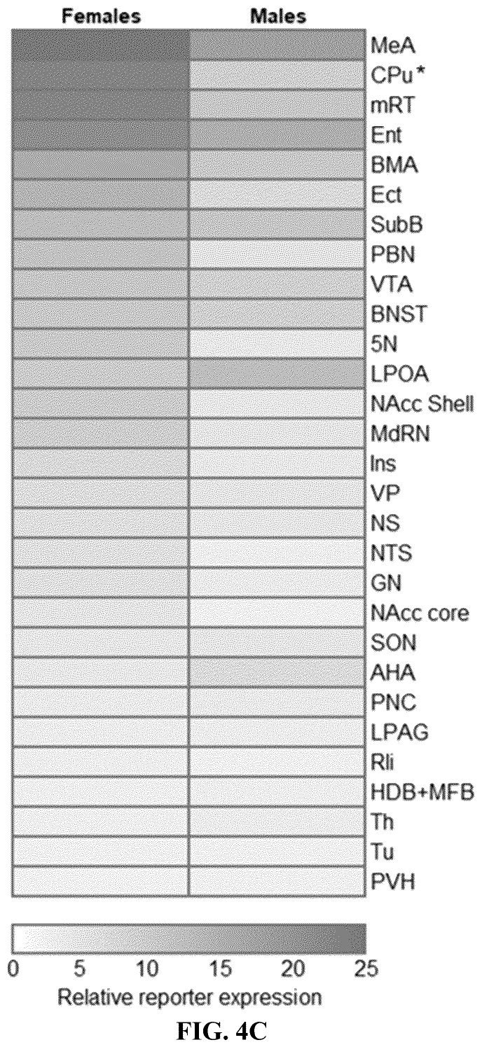

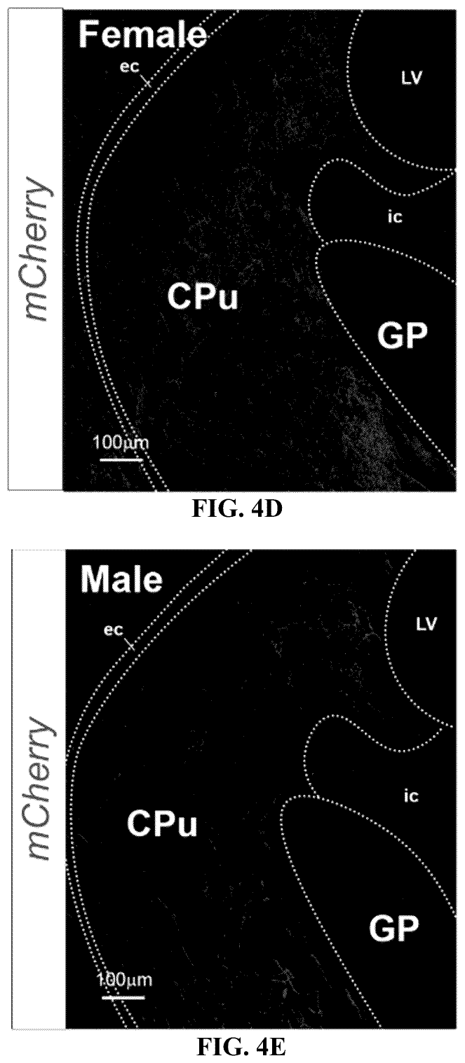

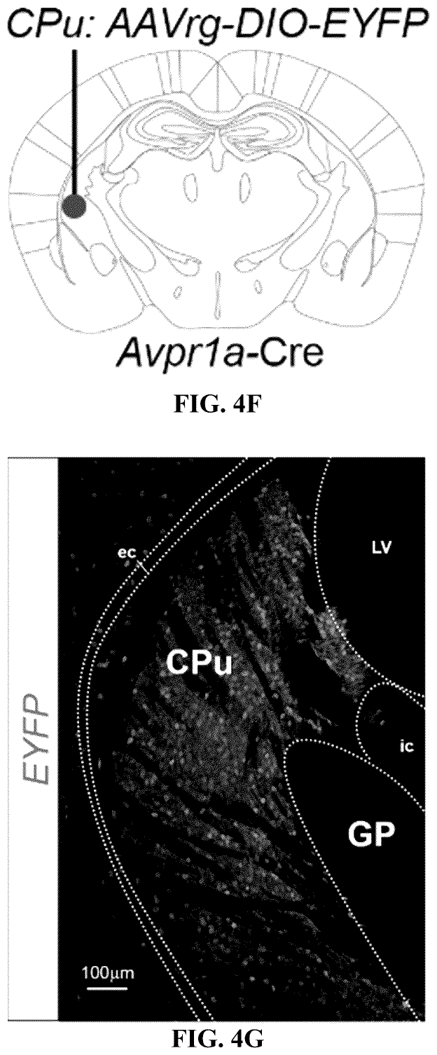

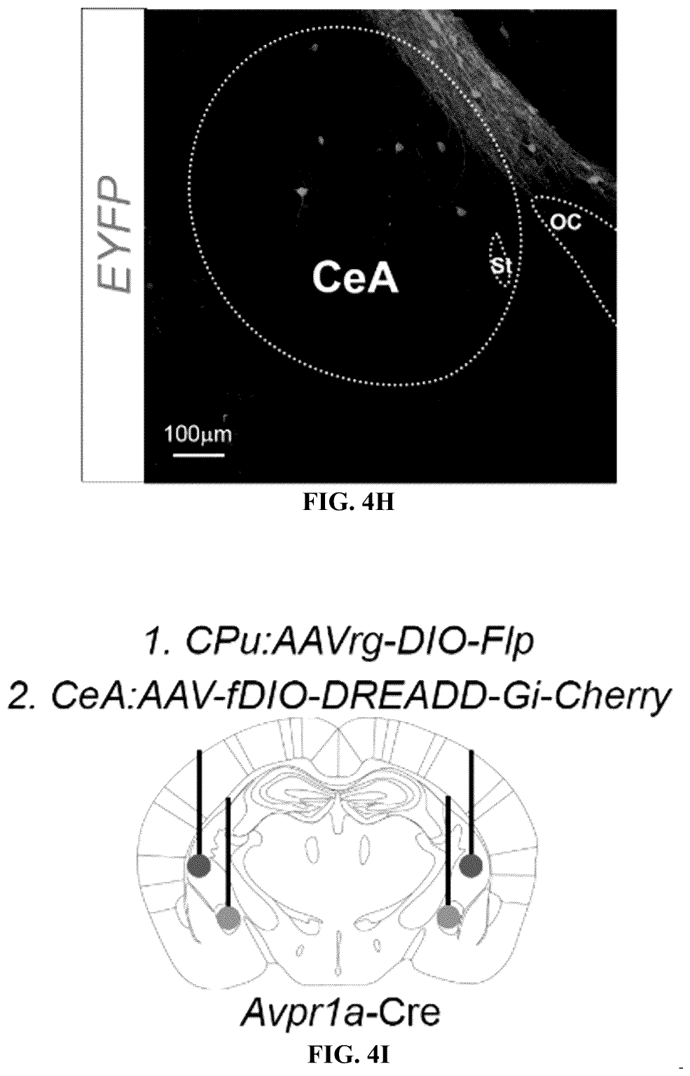

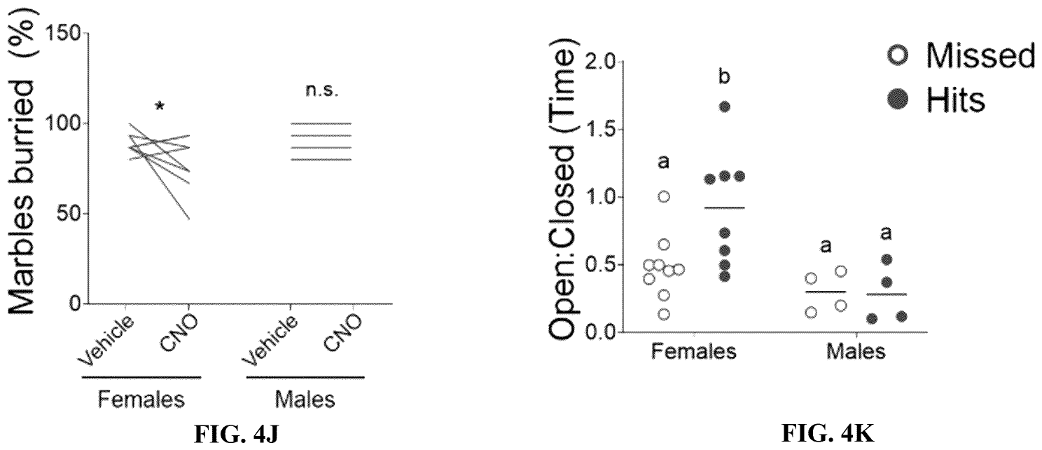

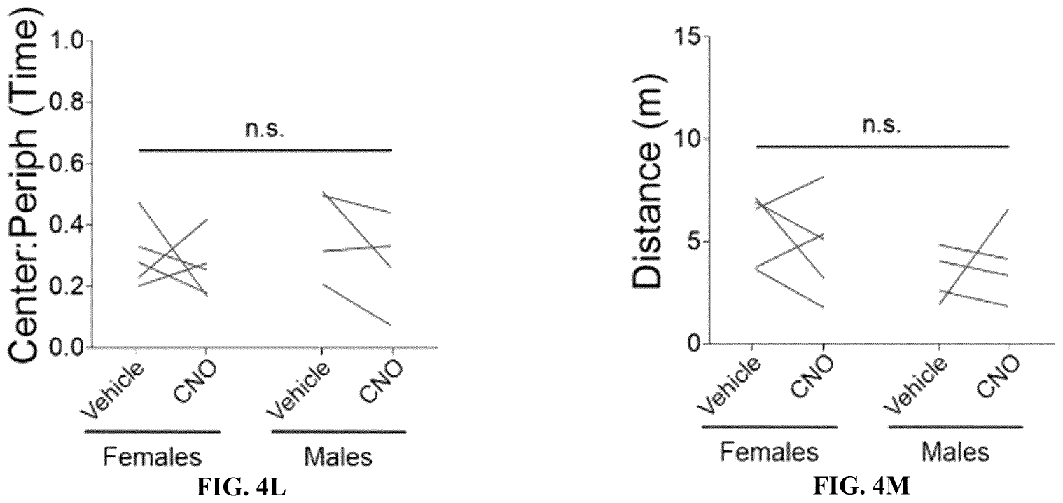

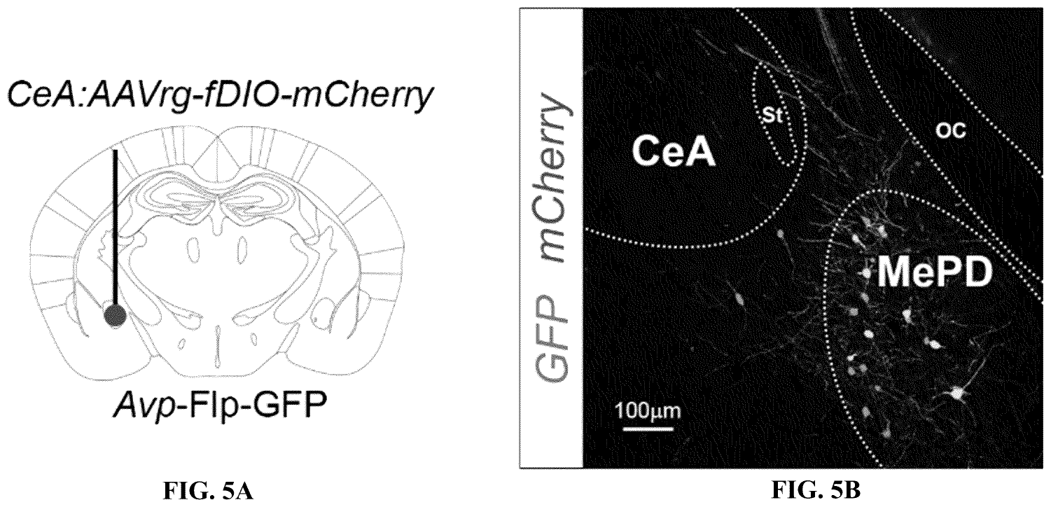

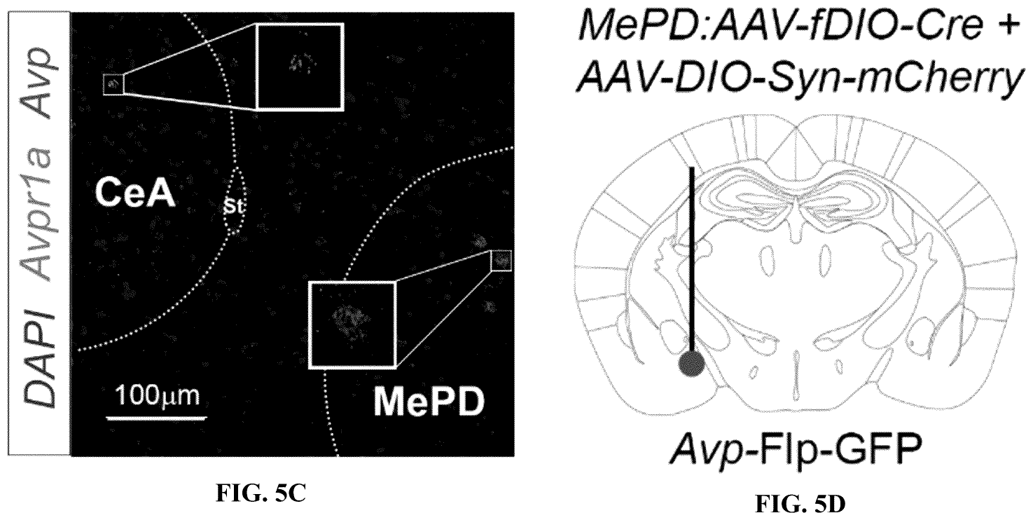

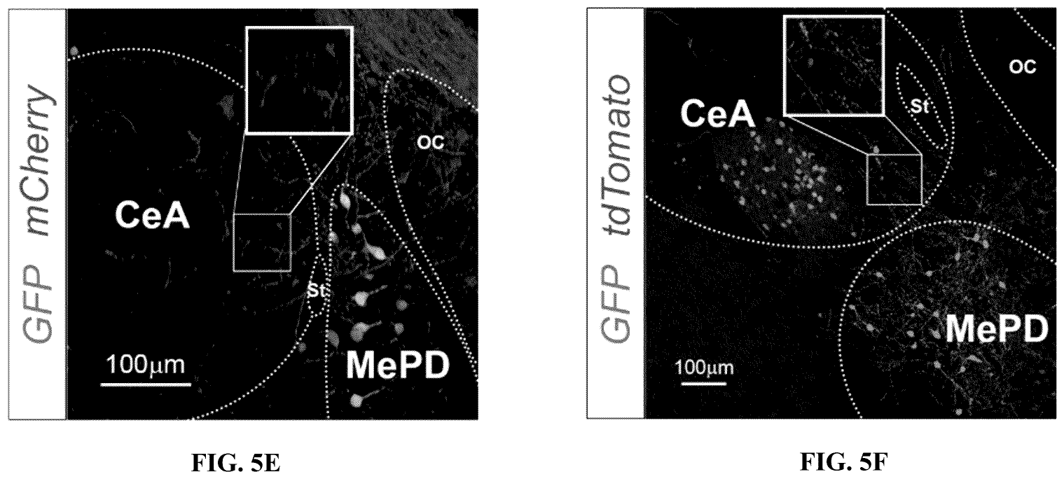

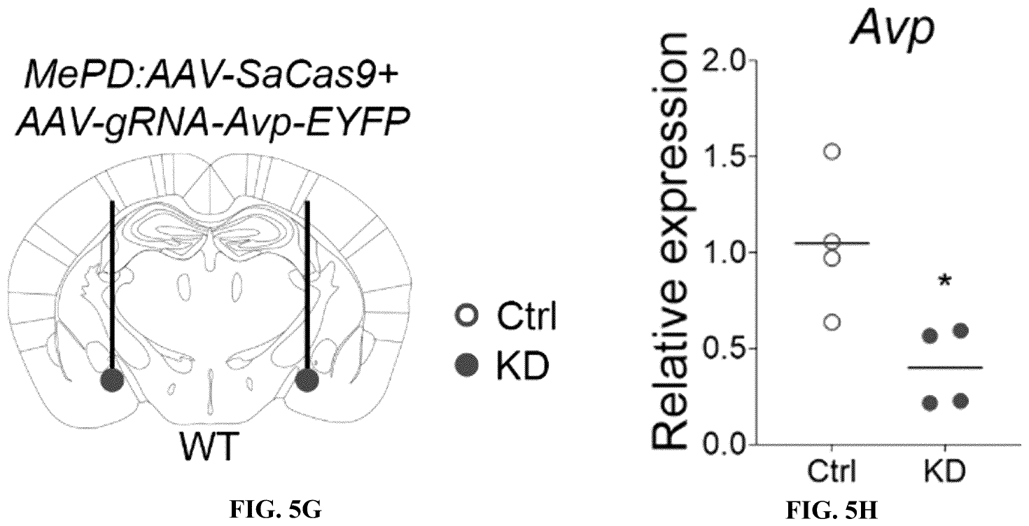

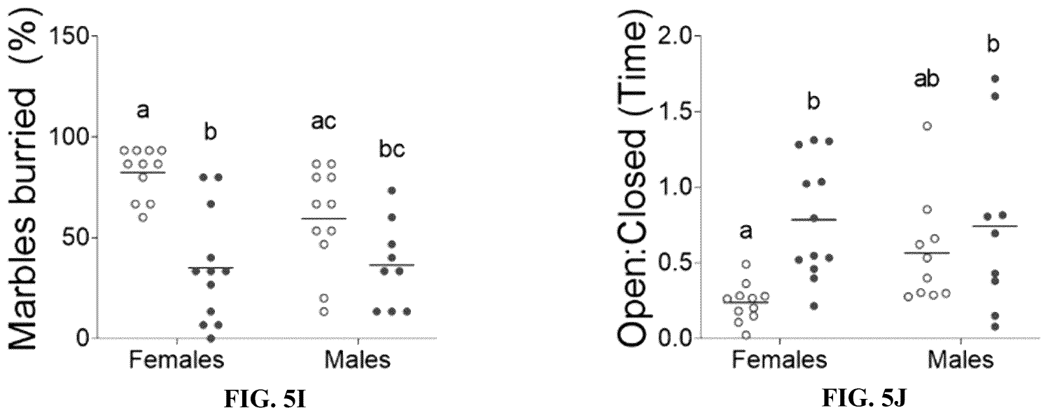

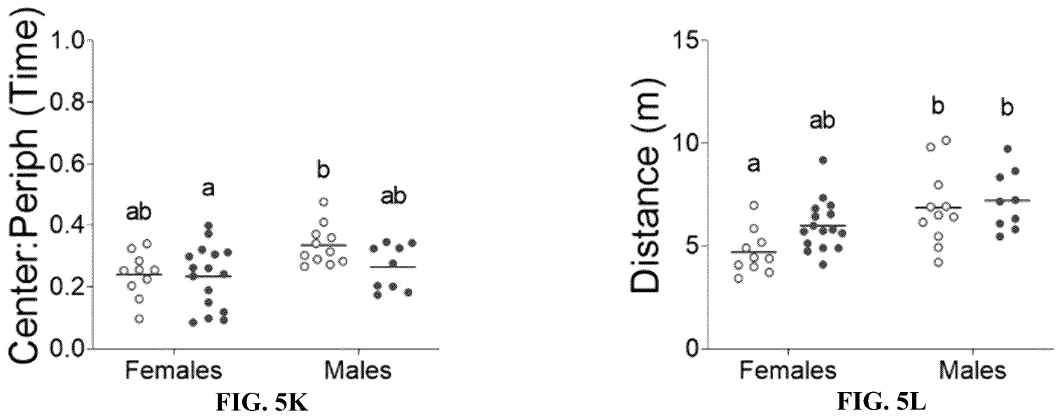

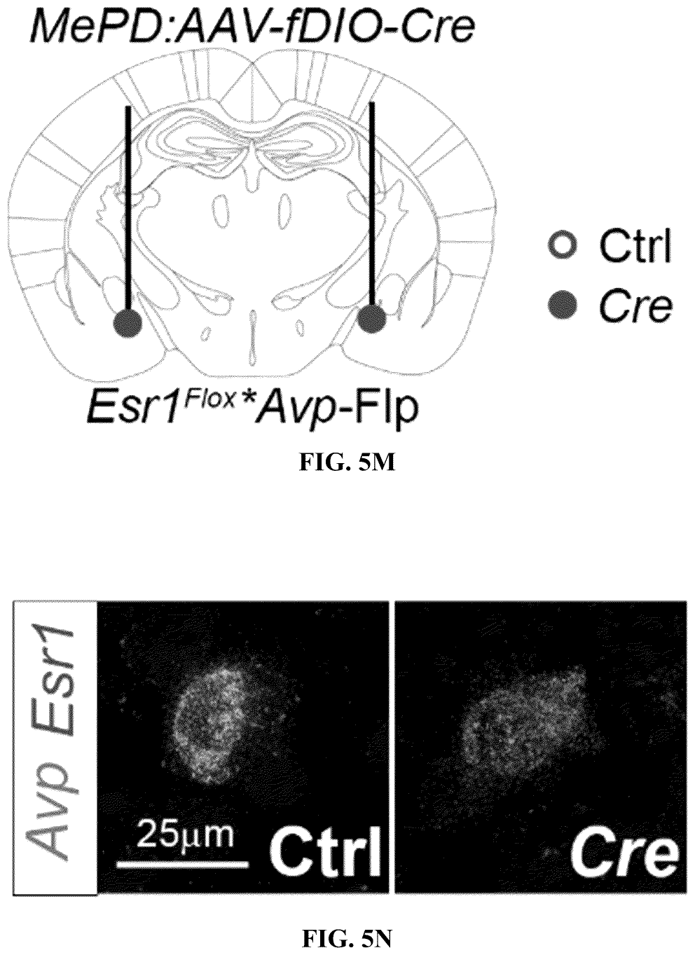

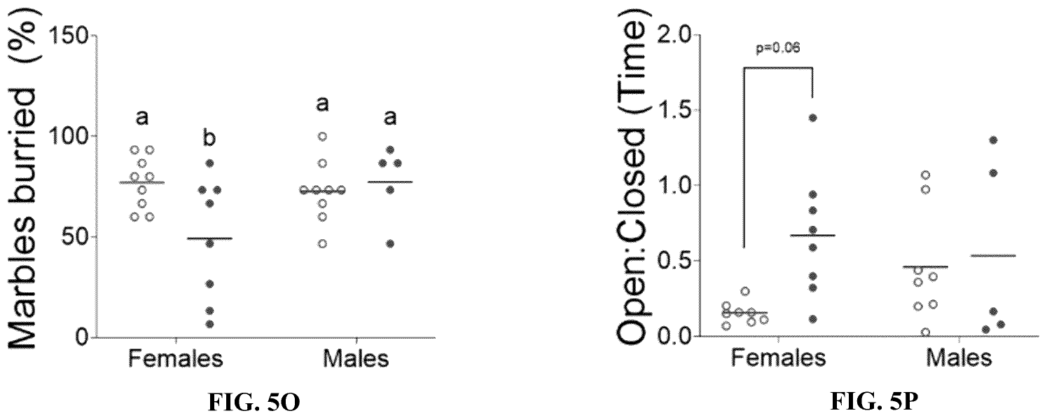

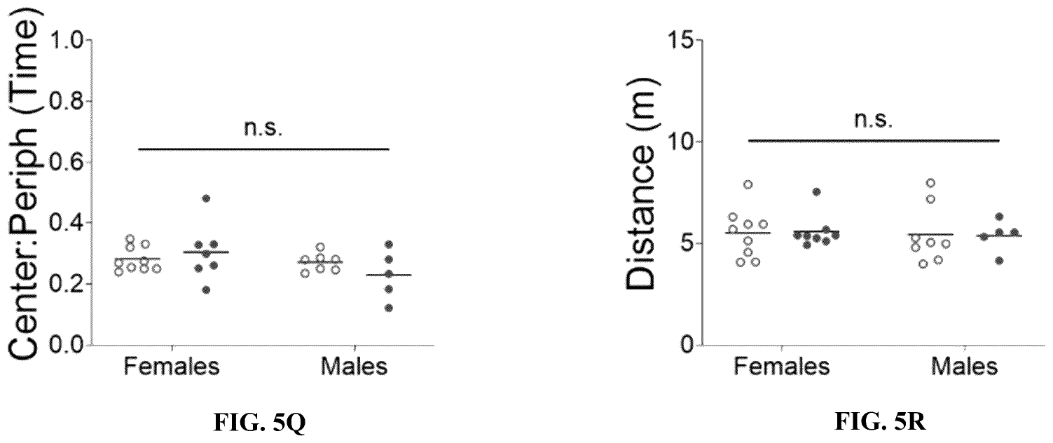

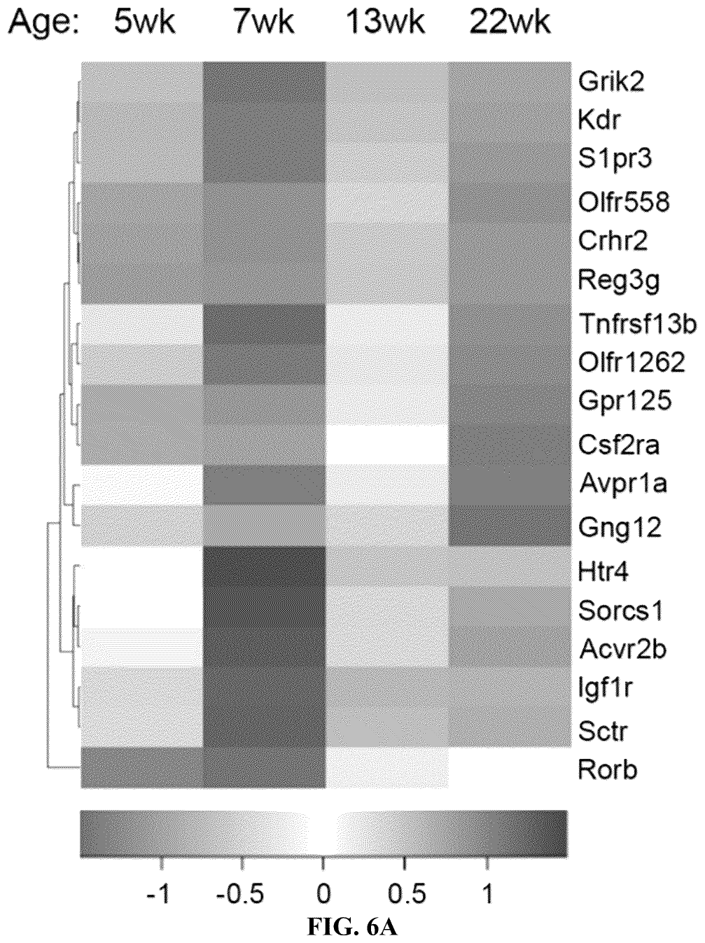

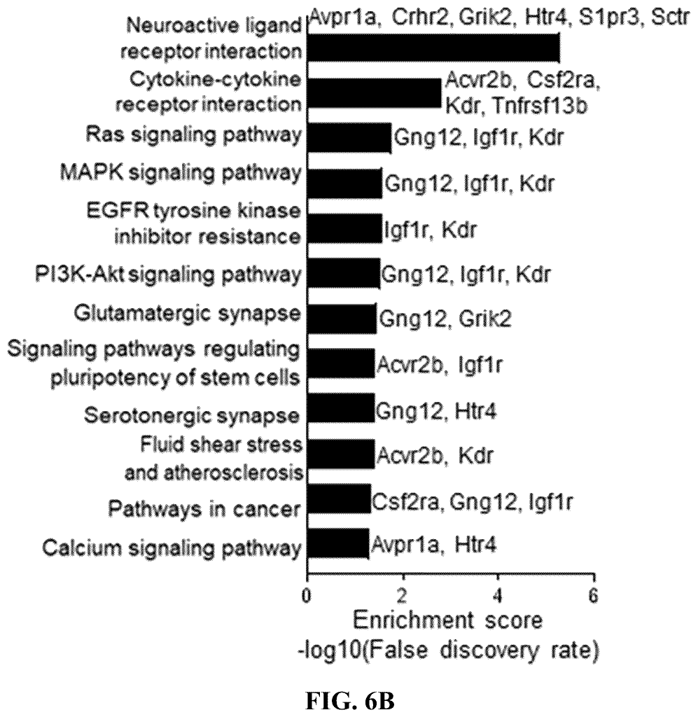

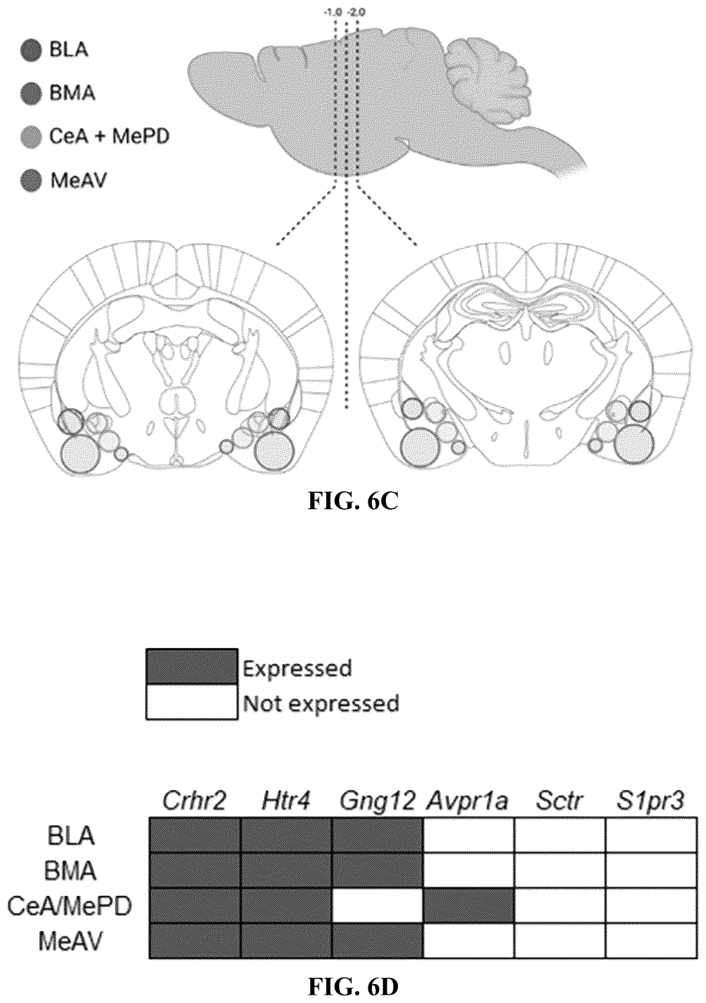

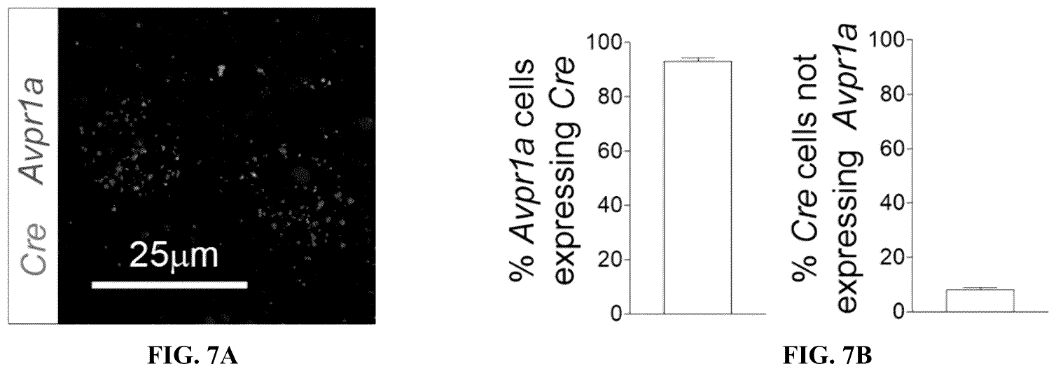

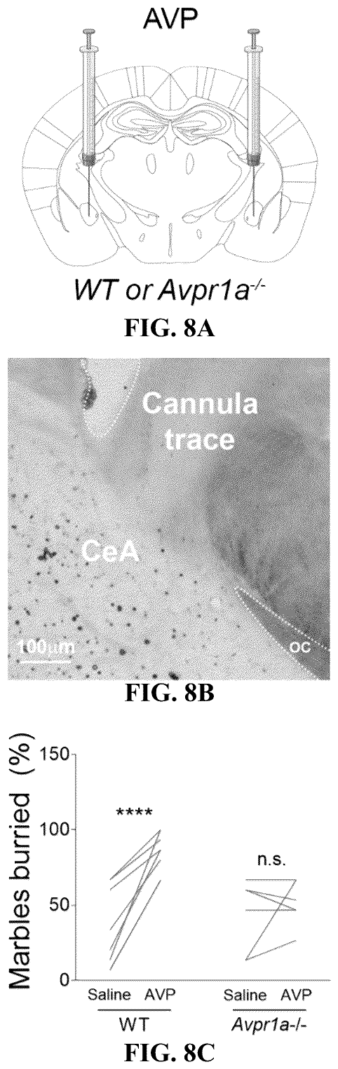

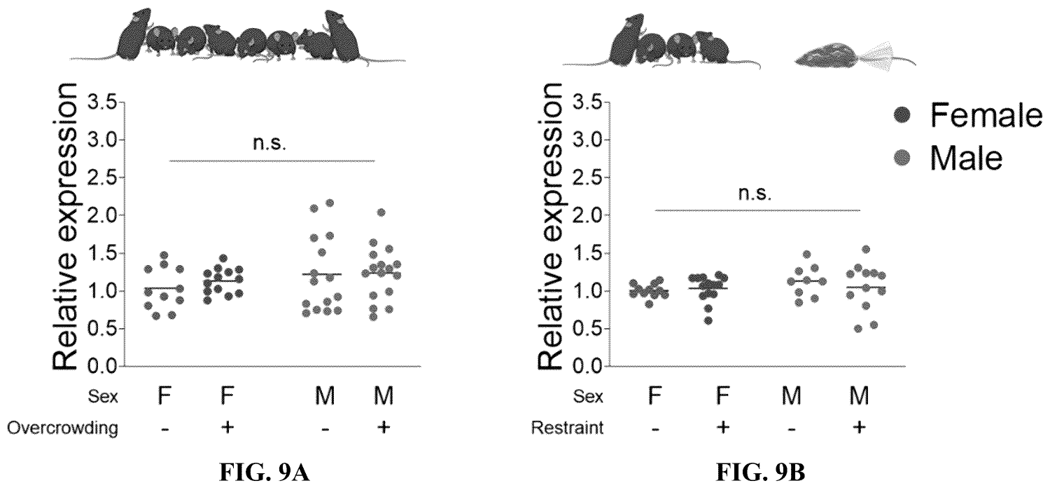

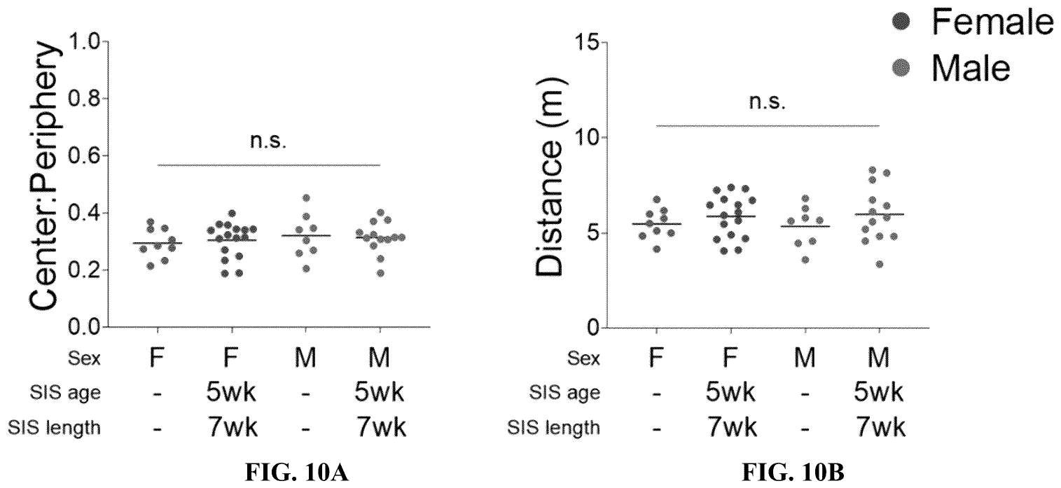

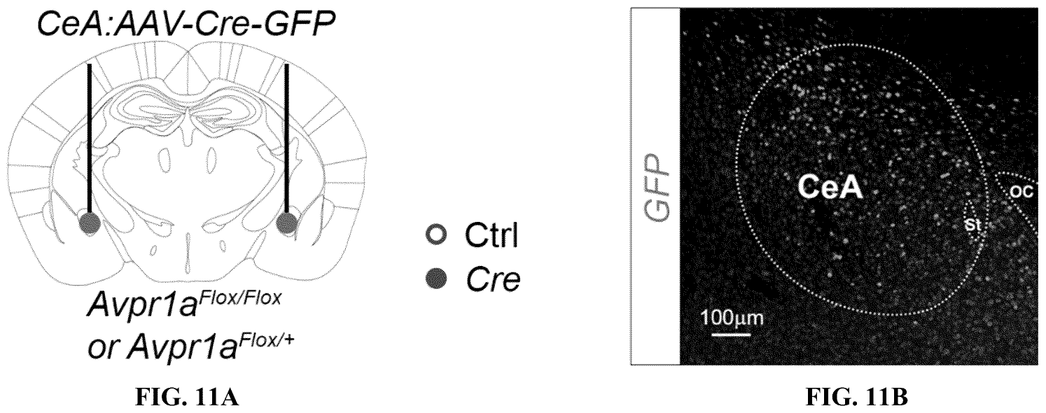

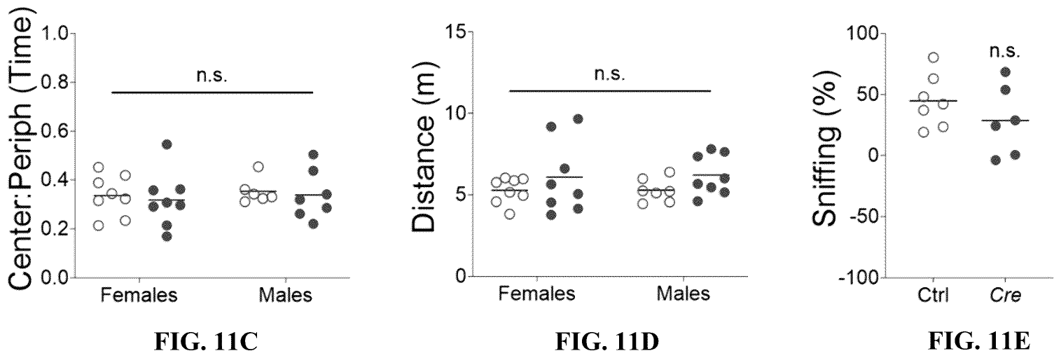

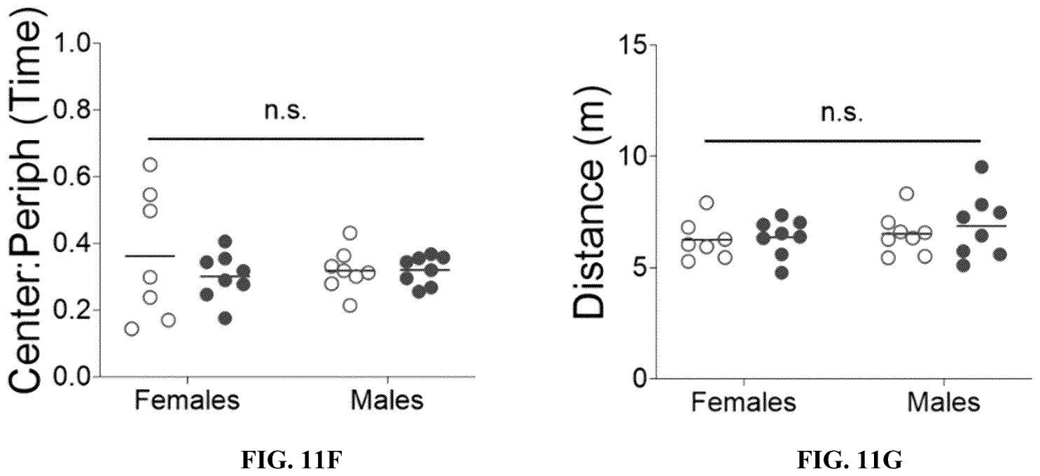

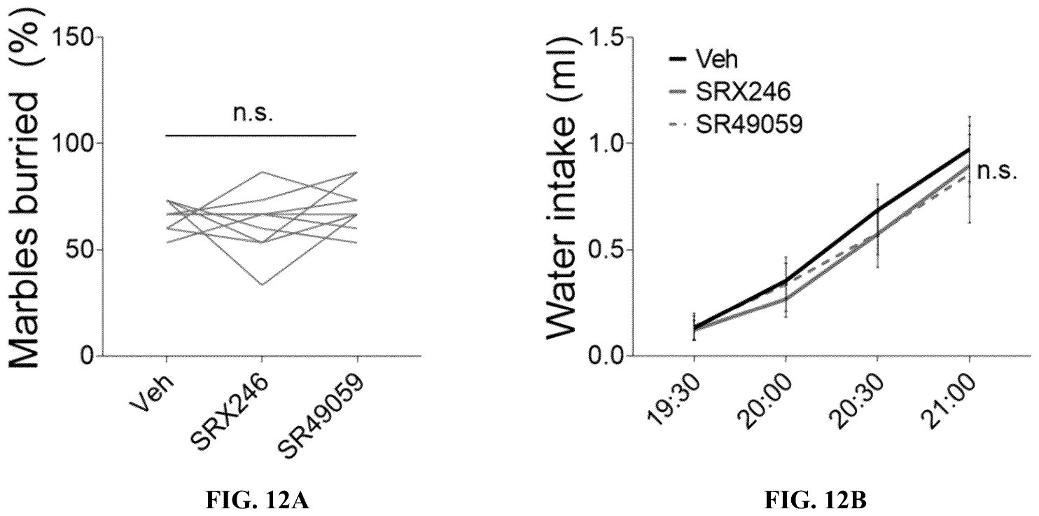

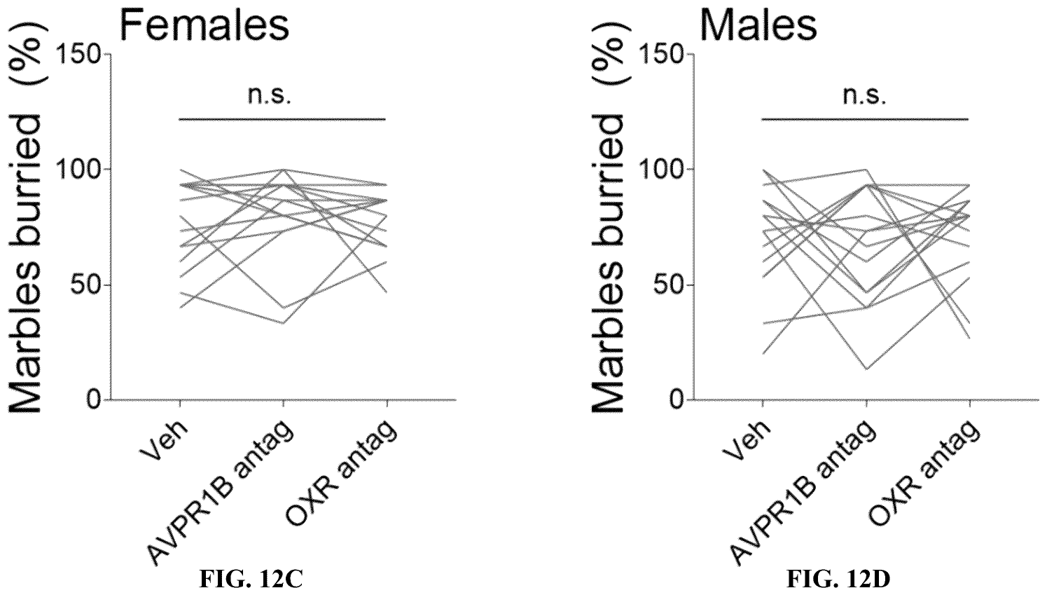

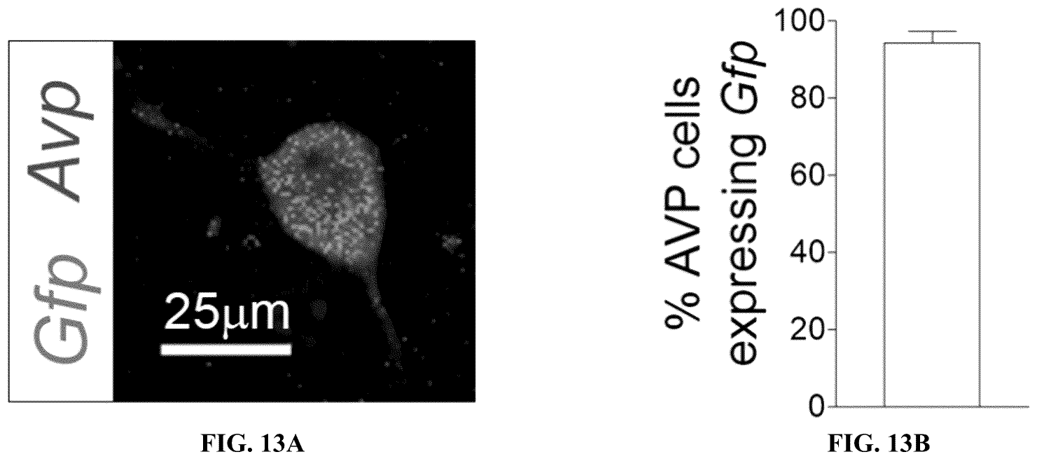

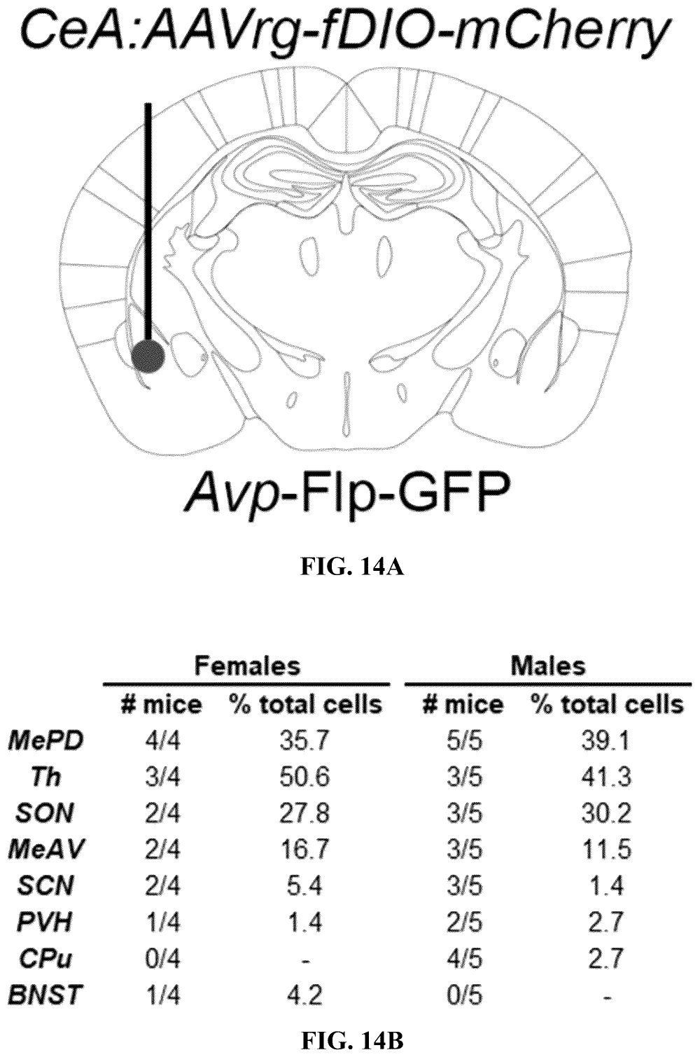

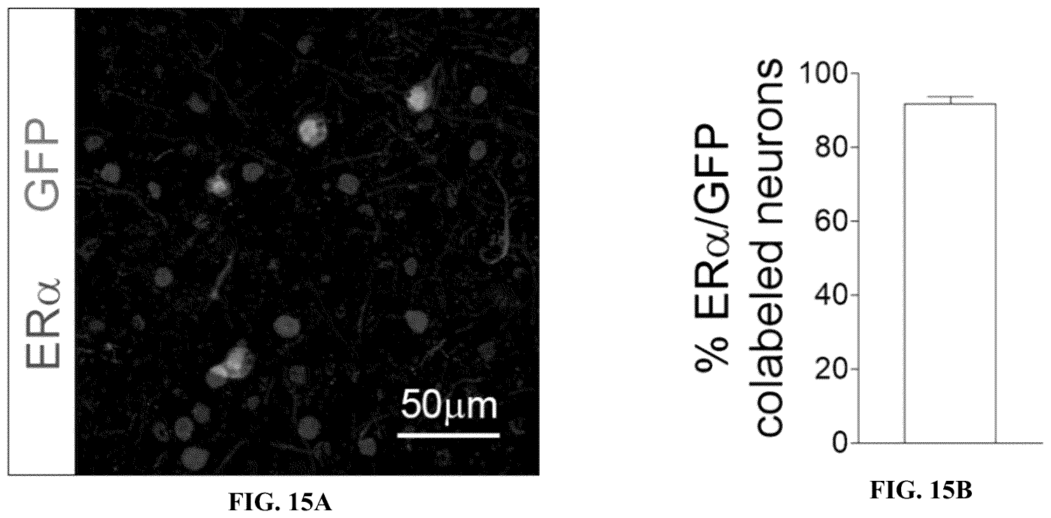

FIG. 6A-FIG. 6D (Supplemental FIG. 1¹) ¹ References to Supplemental Figures refer to the parallel figures included in U.S. Provisional Application No. 63/485,248, filed Feb. 15, 2023.

(A) Heat map of the expression of the 15 genes upregulated at 7 weeks that were classified as having “molecular transducer activity” by Gene Ontogeny (n=3). (B) KEGG analysis for the genes upregulated at 7 weeks within the molecular transducer activity family. (C) Schematic of micropunches in the amygdala used for quantification by qPCR. (D) Summary of qPCR analyses to characterize expression of genes encoding GPCRs of interest in subregions of the amygdala.

FIG. 7A-FIG. 7B (Supplemental FIG. 2)

(A) Co-expression of Avpr1a and Cre mRNA with smFISH in coronal sections of the CeA. (B) Quantification of the extent of Avpr1a and Cre co-expression in the CeA, as detected by smFISH (n=6).

FIG. 8A-FIG. 8B (Supplemental FIG. 3)

(A) Schematic of bilateral AVP injections in the CeA of cannulated WT or Avpr1a^{-/-} littermates. (B) Representative image of the cannula trace in coronal sections of the CeA. (C) Effect of bilateral AVP injections in the CeA on marble burying in female WT vs Avpr1a^{-/-} littermates (n=6-8).

FIG. 9A-FIG. 9B (Supplemental FIG. 4) (A-B) Expression of Avpr1a in the CeA of mice exposed to social overcrowding (n=11-16) (A) or repeated restraint stress (n=9-14) (B).

FIG. 10A-FIG. 10B (Supplemental FIG. 5)

(A-B) Effect of CSIS on time spent in the center of the open field (A), and distance traveled in the open field (B) in WT mice (n=7-16).

FIG. 11A-Fig. G (Supplemental FIG. 6)

(A) Schematic of bilateral injections of AAV-Cre-GFP (closed circles) vs. AAV-GFP controls (open circles) in the CeA of mice carrying two vs. one floxed allele of Avpr1a that were exposed to CSIS. (B) Expression of the viral GFP reporter in coronal sections of the CeA. (C-E) Effect of CeA Avpr1a deletion on time spent in the center of the open field (C), distance traveled in the open field (D), and social interaction (E) in Avpr1a^{Flox/Flox} homozygotes exposed to CSIS (n=7-11). (F-G) Effect of CeA Avpr1a deletion on time spent in the center of the open field (F), and distance traveled in the open field (G) in Avpr1a^{Flox/+} heterozygotes exposed to CSIS (n=7-11).

FIG. 12A-FIG. 12D (Supplemental FIG. 7)

(A) Effects of i.p. injections of AVPR1A antagonists, SRX246 and SR49059, on marble burying in female mice housed in groups of 4-5 (n=9). (B) Effects of i.p. injections of SRX246 and SR49059 on water intake in female mice exposed to CSIS (n=8-10). (C) Effects of i.p. injections of AVPR1B and oxytocin receptor antagonists on marble burying (15 min) in female mice exposed to CSIS (n=15). (D) Effects of i.p. injections of AVPR1B and OXR antagonists on marble burying (15 min) in male mice exposed to CSIS (n=15).

FIG. 13A-FIG. 13B (Supplemental FIG. 8)

(A) Co-expression of Avp and Gfp in the MePD of Avp-Flp::GFP mice detected with smFISH. (B) Quantification of Avp-expressing neurons that co-express Gfp in the MePD (n=3).

FIG. 14A-FIG. 14B (Supplemental FIG. 9)

(A) Schematic of retrograde tracing of AVP neurons that project to the CeA with unilateral AAV-fDIO-mCherry injections in the CeA of Avp-Flp-GFP mice. (B) Summary of brain regions with neurons labeled with both the GFP AVP lineage trace and the viral mCherry reporter, number of mice where GFP and mCherry co-expression was observed, and percentage of GFP-expressing cells that were labeled with the mCherry reporter within each brain region in females and males (n=4-5).

FIG. 15A-FIG. 15B (Supplemental FIG. 10)

(A) Co-expression of ERα and the GFP lineage trace in AVP^{MePD} neurons, detected with immunohistochemistry in coronal sections of the amygdala. (B) Quantification of the percentage of ERα neurons that co-express the AVP lineage trace (GFP) in the MePD (n=4).

### DETAILED DESCRIPTION OF THE EMBODIMENTS

One embodiment of the present subject matter provides a compound useful for treating anxiety or obsessive-compulsive disorder associated with social isolation in a female subject, wherein the composition comprises a compound that blocks or reduces AVPR1A signaling in the amygdala of the subject. In another embodiment, the female subject is then-currently experiencing social isolation. In another embodiment, the compound comprises an AVPR1A antagonist, such as, for example, SRX246. In another embodiment, the compound blocks or reduces AVPR1A signaling in the central nucleus of the amygdala (CeA) of the subject.

Another embodiment provides a method of administering an effective amount of any of these compositions in a method for blocking or reducing AVPR1A signaling in a female subject in order to treat or reduce anxiety or obsessive-compulsive disorder associated with social isolation.

Another embodiment provides a method of treating or reducing anxiety or obsessive-compulsive disorder associated with social isolation in a female subject, comprising administering to the female subject a composition comprising an effective amount of a compound blocking or reducing AVPR1A signaling in the subject. In one embodiment, the female subject is then-currently experiencing social isolation. In another embodiment, the compound comprises an AVPR1A antagonist. In another embodiment, the compound blocks or reduces AVPR1A signaling in the central nucleus of the amygdala (CeA) of the subject. In a further embodiment, the compound comprises an AVPR1A antagonist. In another embodiment, the composition comprises SRX246.

### OBSERVATIONS

We find that social isolation results in upregulation of AVPR1A in the CeA of females only. We also find that the anxiolytic effects of SRX246 are much stronger in socially isolated females vs. males. This drug does not affect anxiety-like behavior in group-housed females.

AVP is traditionally viewed as a “male” hormone that promotes aggression and/or anxiety. Conversely, oxytocin is viewed as the female counterpart. In a surprising development, we identified AVPR1A as a therapeutic target for social isolation-induced anxiety in females.

We observed the following sex-specific results regarding only females:
Prolonged social isolation is associated with increased anxiety-like behaviors (marble burying and elevated plus assays) in females. Socially isolated males increased marble burying (an OCD-like behavior) but did not alter anxiety-like behavior in the elevated plus maze.
Social isolation leads to increased AVPR1A expression in the amygdala of females, but not males.
Genetic deletion of AVPR1A in the amygdala protects against social isolation-induced anxiety in females but did not provide similar results in males.
Peripheral delivery of an AVPR1A antagonist reverses social isolation-induced anxiety in females but did not provide similar results in males.

Accordingly, compounds that block or reduce AVPR1A signaling may be used to treat anxiety or obsessive-compulsive disorder in socially-isolated females. These studies incorporated issues of chronic isolation; sex specificity; and active time constraints.

### Chronic Social Isolation

Chronic social isolation-exemplified over a 7-week period-increases anxiety-like behaviors in females and expression of AVPR1A in the central nucleus of the amygdala.

Genetic and pharmacological approaches to block AVPR1A signaling reverse anxiety-like behaviors induced by chronic social isolation but have no effect on group-housed females.

### Sex Specificity

Chronic social isolation increases anxiety-like behaviors in females and not males. The AVP system has previously been studied most in the context of aggression and anxiety-like behavior in males. Here, we show that AVPR1A signaling is elevated in chronically socially isolated females, and not males. Accordingly, genetic and pharmacological approaches to block AVPR1A signaling reverse anxiety-like behaviors induced by chronic social isolation in females but have no effect in males.

We note that artificial activation of AVPR1A circuits in the amygdala (by injecting AVP or chemogenetic approaches to stimulate AVPR1A neurons) can induce anxiety-like behaviors in males. This suggests that the circuit is conserved between males and females, but the engagement of the AVPR1A system is unique to females.

### Time Constraints—Mice are Nocturnal

We observed chronic social isolation-induced anxiety in female mice when the behavioral assays are performed at the start of the active phase in the dark cycle. This is to be expected, as mice are nocturnal, and thus active at night rather than during the day.

The length of social isolation is also important-social isolation for shorter periods of time, such as for two weeks, does not increase AVPR1A expression. However, elevated AVPR1A expression caused by chronic social isolation is not reversed when female mice are regrouped for 3 weeks.

### DISCUSSION

These studies provide novel insights into the mechanism underlying sex differences in susceptibility to chronic social stress, a major risk factor for anxiety disorders (Brown, 1993). We found that exposure to CSIS after puberty, which deprives females of preferred affiliative relationships (Palanza, 2001), leads to sex-specific anxiety-related behavioral adaptations. We identified an estrogen-sensitive AVP→AVPR1A circuit in the amygdala that is necessary and sufficient to mediate the sexually dimorphic behavioral responses to CSIS.

### Identification of Amygdala AVPR1A as a Key Mediator of Sex-Specific Responses to CSIS

We initially identified Avpr1a as a gene whose expression is elevated in the female amygdala during the reproductive period, and is increased in response to CSIS, but not social overcrowding. Targeted loss of Avpr1a in the amygdala abrogates the effects of CSIS on adaptive behaviors in the EPM and marble burying assays exclusively in females. While sex differences in the AVP system (DeVries et al., 1985), and links to anxiety and aggression in humans and rodents, are well-documented (Beiderbeck et al., 2007; Bredewold and Veenema, 2018; Coccaro et al., 1998; Murgatroyd et al., 2004), the prevailing idea is that it acts primarily in males. Moreover, congenital global loss of Avpr1a led to deficits in social recognition and anxiety-related behaviors in males and not females (Bielsky et al., 2004; Bielsky et al., 2005b).

AVPR1A circuits in the brain mediating distinct complex behaviors are differentially sensitive to the timing of the stress exposure and the type of stress involved. Studies of the HPA axis in the context of maternal separation during lactation provided the first evidence of sex-specific effects of stress on the AVP system (Veenema et al., 2006; Veenema et al., 2007). Studies involving targeted delivery of antagonists support a role for AVPR1A in widely distributed brain regions that regulate different behaviors. AVPR1A in the PVH enhances maternal care and increases anxiety-related behaviors in lactating females (Bayerl et al., 2016). AVPR1A in the lateral septum (LS) regulates social recognition and play behavior in a sex-specific manner and is sensitive to exposure to acute novel environmental stress after puberty (Bielsky et al., 2005a; Bluthe and Dantzer, 1990; Bredewold et al., 2014; Dantzer et al., 1988; Everts and Koolhaas, 1999; Veenema et al., 2012). In the medial preoptic area and anterior hypothalamus of adult males, AVPR1A promotes a scent marking behavior involved in social communication (Albers et al., 1986), while it acts in the MeA to drive avoidance of an odor associated with sickness (Arakawa et al., 2010).

Here, targeted loss of Avpr1a experiments provide strong evidence that the CeA plays a critical role in mediating effects of CSIS in post-pubertal females and not males. Moreover, we found that AVPR1A antagonists had no effect on behavior in group-housed females tested at the onset of the dark phase, which likely explains the failure to observe effects of global loss of Avpr1a function on anxiety-related behaviors in group-housed females that were tested in the first half of the light phase (Bielsky et al., 2005b). In the context of CSIS, the timing of exposure impacts the type of circuits and behaviors affected. Imposing CSIS at weaning led to increased social (aggressive) behaviors that were associated with changes in AVPR1A binding in the LH, DG, and BNST, while anxiety-related behaviors and binding in the CeA were not altered (Oliveira et al., 2019).

Not only is the timing of social isolation critical to engage AVPR1A circuits in the amygdala, but so is the duration of exposure, as we found that Avpr1a expression was not significantly increased in females singly housed for 2 weeks. In summary, we provide the first evidence that AVPR1A^{CeA} circuits promote adaptive anxiety-related behaviors in females in response to CSIS in post-pubertal females, conditions that capture many of the features of social exclusion and loneliness (Cacioppo et al., 2015; Palanza, 2001) but are rarely examined in rodent models.

### Avpr1a-Expressing Neurons in the CeA Act in a Distributed Network to Induce Anxiety-Related Behaviors.

AVPR1A is expressed in a small population of neurons in the medial-most aspect of the CeA that projects most strongly to sites in the amygdala, forebrain and midbrain reticular formation that regulate goal-directed behaviors, habit formation and arousal (Azzopardi et al., 2018; Knowlton et al., 1996; Lingawi and Balleine, 2012; Seiler et al., 2022; Smith and Graybiel, 2013; Yin and Knowlton, 2006). This contrasts with the remainder of the CeA, which sends descending projections to midbrain and brainstem circuits that regulate appetitive, aversive, and defensive behaviors (Kim et al., 2017; Torruella-Suarez et al., 2020; Tovote et al., 2016; Wang et al., 2023). Other groups reported that activation of subpopulations of CeA neurons can also induce anxiety-related behaviors. Optogenetic stimulation of CeA→basolateral amygdala circuits can induce these behaviors (Tye et al., 2011), but based on our tracing studies, these are distinct from AVPR1A^{CeA} neurons. Chemogenetic inhibition of CeA→BNST circuits prevents anxiety-related behavioral adaptations in the context of sepsis (Bourhy et al., 2022). Since they did not target their manipulations to a genetically defined subpopulation of neurons, it is possible that some AVPR1A^{CeA} neurons contributed to this effect. Chemogenetic activation of CeA neurons expressing Crhr1 (Weera et al., 2022) or Tac2 (Zelikowsky et al., 2018) can also modulate anxiety-related behaviors. However, since these genes are expressed in many CeA neurons, including some that regulate aggression and defensive (Zelikowsky et al., 2018) or nociceptive (Weera et al., 2022) behaviors, it is possible that there is some contribution from AVPR1A^{CeA} neurons. These observations highlight the importance of identifying neurons that respond to different types of endogenous stressors rather than “anxiety-related” behavioral outcomes.

We focused on the CPu, because it was the only region where we detected significantly more AVPR1A^{CeA} projections in females. The CPu has been implicated in the physiopathology of anxiety disorders (Lago et al., 2017). Inhibition of AVPR1A^{CeA}→CPu circuits reversed CSIS-induced anxiety-related behavioral adaptations in females and not males, supporting the idea that they play an important role in mediating these effects. While gain of function approaches induce anxiety-related behaviors, the sex-specificity is lost. We observed that intra-CeA AVP injections in female WT mice trigger anxiety behavior in WT females, but not in global Avpr1a^{-/-} knockouts. Exogenous delivery of AVP to the CeA was also shown to increase anxiety-related behaviors in males (Hernandez et al., 2016). Moreover, chemogenetic activation of AVPR1A^{CeA} neurons was sufficient to induce anxiety-related behaviors in males and females. Together, these data support the idea that AVPR1A^{CeA} circuits can modulate anxiety-related behaviors in both sexes, but in the context of post-pubertal CSIS they are only engaged in females. It is possible that AVP→AVPR1A circuits in the amygdala are preferentially activated in males in other contexts, such as social defeat stress in adulthood (Barchiesi et al., 2021).

### ERα Signals in AVP^{MePD} Neurons Mediate Sex-Specific Effects of Post-Pubertal CSIS on Anxiety-Related Behavioral Adaptations.

AVP neurons are distributed throughout the brain and their projection patterns are notable for their high degree of sexual dimorphism (De Vries et al., 1994a). These neurons are also responsive to gonadal hormones (Brot et al., 1993; De Vries et al., 1994b; Shapiro et al., 2000; Somponpun and Sladek, 2002; van Leeuwen et al., 1985; Vilhena-Franco et al., 2019), supporting the idea that AVP plays an important role in mediating sex differences in behavior. Based on tracing studies in male rats, it has been assumed that the PVH is the primary source of AVP to the CeA (Hernandez et al., 2016). We did not observe robust AVP projections from the PVH in males or females, consistent with studies in humans (Sivukhina and Jirikowski, 2021). Instead, we identified the MePD as a major source of AVP to the medial-most portion of the CeA. AVP^{MePD} neurons projected in the vicinity of AVPR1A^{CeA} neurons in both males and females but did not contact them directly, consistent with a paracrine mode of release of AVP^{MePD} neurons (Landgraf and Neumann, 2004).

Regulation of AVP expression and release from the MePD by gonadal hormones is well-documented, but studies were almost exclusively conducted in males (De Vries et al., 1994b; Plumari et al., 2002; Scordalakes and Rissman, 2004; Wang, 1994; Wang and De Vries, 1995). Chronic depletion of estrogen resulted in a marked decreased in AVP (immunoreactivity) in the MePD of males; females were not assessed (Plumari et al., 2002). This effect was dependent on the expression of both androgen receptors and ERα, as loss of only ERα had no effect (Scordalakes and Rissman, 2004). Here, knockdown of Avp in the MePD and loss of Esr1 from AVP^{MePD} neurons decreased some of the CSIS-induced behavioral adaptations in females, consistent with a sex-specific role for Era signals in promoting AVP expression and or release.

### Potential Therapeutic Implications

Loneliness is widespread and has detrimental effects on health and quality of life (House et al., 1988). Our finding that AVP^{MePD}→AVPR1A^{CeA} circuits mediate effects of CSIS on anxiety-related behavioral adaptations in female mice raises the possibility that they also contribute to the heightened susceptibility of women to social exclusion and loneliness (Cacioppo et al., 2015; Palanza, 2001). A growing body of evidence supports the idea that social restrictions and other lockdown measures established to control COVID-19 outbreaks had unanticipated adverse effects on the mental health of young adults (Klaser et al., 2021; Taquet et al., 2021). The pandemic caused an estimated 76.2 million additional cases of anxiety disorders across the globe, particularly in young adult females (Collaborators, 2021; Klaser et al., 2021).

There is empirical support for the use of pharmacotherapies in patients (subjects) with anxiety disorders, but many do not respond to treatment (Taylor et al., 2012). These drugs were developed for other disorders and act on a wide range of receptors that are broadly expressed in the central and peripheral nervous systems (e.g., serotonin, dopamine, adrenergic and GABA receptors) (Szuhany and Simon, 2022). Targeting AVPR1A circuits that respond to the removal of affiliative relationships in postpubertal female mice diminished anxiety-related adaptive behaviors. Avpr1a is expressed in the human amygdala (Herrero et al., 2020). This raises the possibility that AVPR1A antagonists that have been proven to be safe in clinical trials, such as SRX246 (Brownstein et al., 2020), could be effective treatments for anxiety associated with social exclusion or loneliness in women. Since we found that SRX246 did not affect anxiety-related behaviors in males or group-housed females, consideration of sex and perceived loneliness should be used to identify people who are more likely to respond to treatment. In conclusion, our studies are consistent with the growing call to develop therapeutics that target the etiology of a psychiatric disorder, rather than its symptoms.

### EXAMPLES

### Example 1

### Identification of Genes Upregulated in the Female Amygdala.

We set out to identify genes whose expression in the female amygdala mirrors the period of susceptibility to anxiety disorders (Collaborators, 2021; Craske, 2003; Kessler et al., 2012; Pine et al., 1998; Wittchen et al., 1998). To this end, we generated high-throughput RNA-sequencing profiles of the whole amygdala of group housed female C57BL6/J wild type (WT) mice at 5, 7, 13 and 22 weeks of age. 1,851 genes were identified whose expression increased from 5 to 7 weeks and decreased across adulthood.

We selected the top 300 differentially expressed (DE) genes at 7 weeks (FIG. 1A). The majority (64%) of DE genes belonged to six Gene Ontogeny families: catalytic activity, molecular function regulators, molecular transducers, structural molecules, transcription regulators and transporter activity (http://amigo.geneontology.org/amigo) (FIGS. 1B and 6A). Six genes in the molecular transducer family code for interconnected G-coupled protein receptors (GPCR): Arginine vasopressin receptor 1A (Avpr1a), Corticotropin releasing hormone receptor 2 (Crhr2), Guanine nucleotide binding protein G12 (Gng12), 5-Hydroxytryptamine (serotonin) receptor 4 (Htr4), Sphingosine 1 phosphate receptor 3 (S1pr3) and Secretin receptor (Sctr) (FIG. 1C). Analysis of molecular transducer family genes with the KEGG mapper tool, that identifies receptor-ligand interactions, demonstrated a significant enrichment with the neuroactive ligand-receptor interaction pathway (FIG. 6B).

Next, we characterized the expression patterns of the identified GPCR-encoding genes in subregions of the amygdala by performing RT-qPCR in micropunches. We extracted samples from the anteroventral part of the medial amygdala (MeAV), the basolateral amygdala (BLA), basomedial amygdala (BMA), and a single punch spanning the CeA and MePD (FIG. 6C) in males and females at 7 weeks. Crhr2 and Htr4 expression was detected in all 4 regions examined, Gng12 expression was detected in all punches but the CeA/MePD, while Sctr and S1pr3 transcripts fell below the threshold for detection (average Ct values >31) (FIG. 6D). Avpr1a was the only transcript exclusively found in a single punch, the CeA/MePD (FIG. 6D).

Using single molecule fluorescent in situ hybridization (smFISH), we mapped Avpr1a expression to a small population of GABAergic neurons in the medial-most portion of the CeA, adjacent to the stria terminalis (FIG. 1D). AVPR1A^{CeA} neurons were preferentially located in the caudal CeA between Bregma −1.34 mm to −1.58 mm (FIG. 1E). To better visualize and target this small subpopulation of neurons, we generated an Avpr1a-Cre mouse line and crossed it to a Cre-dependent red fluorescent protein td-Tomato reporter (Avpr1a-Cre::tdTOM) (FIG. 1F). Cre transcript was expressed in over 90% of Avpr1a-expressing cells in the CeA, and in fewer than 10% of Avpr1a-negative cells (FIG. 7).

### Example 2

### Activation of AVPR1A^{CeA} Neurons Elicits Anxiety-Related Behavioral Adaptations in Both Sexes.

Bilateral infusion of AVP into the CeA of male rats elicited anxiety-related behaviors ((Hernandez-Perez et al., 2018; Hernandez et al., 2016). We used the marble burying test to assess the effect of intra-CeA AVP injections in females. This assay takes advantage of the proclivity of rodents to dig in natural settings and in standard cage bedding to assess repetitive, compulsive-like behaviors (Broekkamp et al., 1986).

Intra-CeA injections into group housed WT females increased marble burying but had no effect in global knockouts lacking Avpr1a (Avpr1a^{-/-}) (FIG. 8). We were unable to perform these experiments in group housed males, because aggressive behaviors disrupted the in-dwelling cannulas used to deliver AVP.

We utilized a chemogenetic approach to overcome this technical issue that prevented direct comparisons of males and females. We performed bilateral intra-CeA injections of an adeno-associated virus (AAV) virus expressing a Cre-dependent Designer Receptors Exclusively Activated by Designer Drugs (DREADD)-Gq receptor or a control virus in Avpr1a-Cre mice (FIG. 1G). A single injection of the DREADD ligand, clozapine-N-oxide (CNO, 1.5 mg/kg, i.p.), acutely activated AVPR1A^{CeA} neurons in mice injected with the DREADD-Gq virus but not the control virus (FIG. 1H).

Anxiety-related behaviors were evaluated with the marble burying, elevated plus maze (EPM) and open field tests. The EPM examines the conflict between the drive to explore a new environment and the natural aversion to open spaces (Montgomery, 1958), while the open field test evaluates novelty-induced locomotor behavior as well as approach-avoidance conflict. Meta-analyses support the external validity of the use of the percentage of marbles buried (Langer et al., 2020) and the time spent in the open arms of the EPM (both in absolute terms and as a ratio) (Rosso et al., 2022) to screen for anxiolytic effects.

Chemogenetic activation of AVPR1A^{CeA} neurons elicited anxiety-related behavioral adaptations in both sexes, including increased marble burying (FIG. 1I), and decreased time spent in both the open arms of the EPM (FIG. 1J) and the center of the open field (FIG. 1K). These effects did not result from differences in locomotor activity (FIG. 1L).

### Example 3

### Chronic Social Isolation Stress (CSIS) Leads to Upregulation of Avpr1a Expression in the Female CeA.

AVPR1A in the CeA mediates the heightened sensitivity of females to chronic social stress, a risk factor for anxiety disorders (Brown, 1993). We used social isolation after puberty (5 weeks to ≥12 weeks) in WT female mice to capture the enhanced responsiveness of women to social exclusion (Benenson et al., 2013; Clauss and Byrd-Craven, 2019; Palanza, 2001; Stroud et al., 2002). Avpr1a expression in the CeA was elevated in females, but not males, exposed to CSIS, and not in mice exposed to social crowding or repeated restraint (FIGS. 2A, 9). CSIS also resulted in anxiety-related behaviors in the marble burying assay (FIG. 2B) and the EPM (FIG. 2C) in females but not in males. Time in the center of the open field and locomotor activity were unchanged in either sex (FIG. 10). The effect of CSIS on Avpr1a expression and marble burying behavior persisted even after mice were re-grouped for 3 weeks (FIG. 2E, F).

Next, we explored whether the time of onset or duration of stress impacts Avpr1a expression in the female CeA, since they can influence the nature of behavioral responses (Arakawa, 2018; Bale and Epperson, 2015; Francois et al., 2021; Hodes and Epperson, 2019). Exposure to 7 weeks of social isolation also increased Avpr1a expression when the onset of the stress was delayed from post-puberty (5 weeks) to young adulthood (8 weeks) (FIG. 2D). However, when the duration of adult social isolation was shortened to 2 weeks (10 weeks to 12 weeks), the effect on Avpr1a expression was no longer significant (FIG. 2D). Therefore, when social isolation is imposed after puberty, the length of the exposure is important, while the onset is not.

### Example 4

### AVPR1A Signals are Required for the Effects of CSIS on Anxiety-Related Behaviors in Females.

We used a combination of genetic and pharmacological approaches to test the hypothesis that AVPR1A in the CeA mediates the sex-specific effects of CSIS (from 5 weeks to ≥12 weeks) on anxiety-related behaviors. We generated a new mouse line to conditionally delete Avpr1a (Avpr1a^{flox}).

To validate this model, we performed bilateral injections of AAV-Cre-GFP vs. control AAV-GFP into the CeA of Avpr1a^{flox/flox} homozygotes (FIGS. 2G and 11A and B) and found that Avpr1a expression was reduced by an average of 75% (FIG. 2H). Loss of both copies of Avpr1a decreased marble burying (FIG. 2I) and increased the time spent in the open arms of the EPM (FIG. 2J) in females but not males. Deletion of Avpr1a had no effect on time spent in the center of the open field (FIG. 11C) or on locomotor activity (FIG. 11D). It also did not alter social behaviors, as demonstrated in the social recognition assay in females (FIG. 11E).

Similar to deletion of both Avpr1a alleles from homozygotes, deletion of a single copy from Avpr1a^{flox/+} heterozygotes decreased marble burying in females but not males (FIG. 2K), but it did not affect behavior in the EPM (FIG. 2L), open field test (FIG. 11F) or locomotor activity (FIG. 11G). In summary, CSIS-induced adaptations in anxiety-related behaviors require Avpr1a. Marble burying is most sensitive to this pathway, as deletion of even one copy of the gene was sufficient to block this behavior, while effects in the EPM were only observed when both copies were lost.

We next considered whether acute blockade of central AVPR1A is sufficient to reverse CSIS-induced anxiety-related behaviors. SRX246 is a selective AVPR1A antagonist that can cross the blood brain barrier (Fabio et al., 2012) and has been tested in several Phase II clinical trials (NCT02507284, NCT02733614 and NCT01793441). We assessed the effects of SRX246 (2 mg/kg, i.p.) and an AVPR1A antagonist that cannot cross the blood brain barrier (SR59049, 2 mg/kg, i.p.) in WT mice that were exposed to CSIS starting post-puberty (5 weeks) or in young adulthood (8 weeks).

SRX246 decreased marble burying in females and not males, regardless of the age of CSIS initiation; SR59049 had no effect (FIG. 3A, E). The effect of SRX246 in marble burying assay was specific for CSIS, as it did not change behavior in group-housed females (FIG. 12A). SRX246 also increased time in the open arms of the EPM in females and not males, independent of CSIS onset (FIG. 3B, F). In contrast, water intake, behavior in the open field test and locomotor activity were not affected (FIGS. 3C, D, G, H and 12B). The effect of SRX246 on marble burying in CSIS females was specific to AVPR1A, as injections of AVPR1B (2 mg/kg, i.p.) and OXR (2 mg/kg, i.p.) antagonists did not affect behavior in females or males (FIG. 12C, D). Therefore, acute inhibition of AVPR1A is sufficient to reverse CSIS-induced behavioral adaptations.

### Example 5

### AVPR1A^{CeA}→CPu Circuits Mediate Some of the Behavioral Adaptations to CSIS.

We identified downstream targets of AVPR1A^{CeA} neurons that mediate the effects of CSIS on anxiety-related behaviors. We first performed anterograde tracing by injecting Avpr1a-Cre mice with AAV-DIO-Synaptophysin-mCherry in the CeA (FIG. 4A, B) and quantified the fluorescence intensity in all mCherry-positive regions throughout the brain in both sexes (FIG. 4C). In females, the most prominent projection sites were the medial amygdala (MeA), CPu, mesencephalic reticular formation (mRT) and entorhinal cortex (Ent).

The CPu was the only region with significant more projections in females than males (FIG. 4C-E). To confirm that the CPu is a bona fide downstream target of AVPR1A^{CeA} neurons, we injected a retrograde AAV-DIO-EYFP viral construct in the CPu of Avpr1a-Cre females (FIG. 4F, G) and confirmed that cells in the CeA were labeled with EYFP (FIG. 4H).

Next, we examined whether inhibition of AVPR1A^{CeA}→CPu circuits is sufficient to block CSIS-induced behavioral adaptations. We performed sequential bilateral injections of two viruses in Avpr1a-Cre mice exposed to CSIS: first, a retrograde AAV-DIO-Flp in the CPu, then three weeks later, an AAV-fDIO-DREADDGi-mCherry in the CeA (FIG. 4I). mCherry was expressed in the CeA in only half of the mice injected (“hits”); the remainder did not express mCherry anywhere in the brain (“missed”) and served as controls. Inhibition of AVPR1A^{CeA}→CPu circuits decreased marble burying (FIG. 4J) and increased the time spent in the open arms of the EPM (FIG. 4K) in females but not in males, with no effect on time spent in the center of the open field (FIG. 4L) or on locomotor activity (FIG. 4M). This supports the idea that the CPu mediates some of the sex-specific effects of AVPR1A^{CeA} neurons in the context of CSIS.

### Example 6

### AVP^{MePD} Neurons Project to the CeA.

We used complementary viral tracing approaches to identify sources of AVP to the CeA. We generated an Avp-Flp mouse line that we crossed to a GFP reporter line (Avp-Flp::GFP) and used smFISH to confirm that Gfp transcript was detected in ˜95% of Avp-expressing cells (FIG. 13). First, we identified AVP neurons that project in the vicinity of AVPR1A^{CeA} neurons by injecting Avp-Flp::GFP mice with a retrograde AAV-fDIO-mCherry in the medial aspect of the CeA (FIGS. 5A and 14A). The MePD, as defined in the Paxinos and Franklin Mouse brain atlas (Paxinos and Franklin, 2001), was the only site labeled in 100% of females and males (FIGS. 5B and 14B).

We also observed robust labeling in the thalamus (Th), supraoptic nucleus (SON), anteroventral aspect of the MeA (MeAV) in both sexes, and a few cells the suprachiasmatic nucleus (SCN), paraventricular nucleus of the hypothalamus (PVH), CPu and bed nucleus of the stria terminalis (BNST) (FIG. 14B). We focused on the AVP^{MePD} neurons, which are located in close proximity (˜400 microns) to the AVPR1A^{CeA} neurons (FIG. 5C). AVP^{MePD} neurons account for 35.7-39.1% of all AVP neurons labeled with the retrograde trace (FIG. 14B).

Next, we confirmed that AVP^{MePD} neurons do, in fact, project to the CeA by injecting a mixture of AAV-fDIO-Cre and anterograde AAV-DIO-Synaptophysin-mCherry in the CeA of Avp-Flp::GFP mice (FIG. 5D). We detected mCherry-positive fibers in the medial aspect of the CeA (FIG. 5E). To explore whether AVP^{MePD} neurons send direct projections to AVPR1A^{CeA} neurons, we crossed Avp-Flp::GFP and Avpr1a-Cre::tdTOM mouse lines. While we detected GFP-positive AVP projections in the medial aspect of the CeA, they were not in close contact with AVPR1A^{CeA} neurons (FIG. 5F).

### Example 7

### Knockdown of Avp in the MePD Reverses Effects of CSIS on Anxiety-Related Behaviors in Females.

We examined whether AVP produced in the MePD contributes to CSIS-induced anxiety-related behavioral adaptations. We utilized a virus-based CRISPR approach (AAVs expressing Cas9 and an Avp guide RNA vs. a scrambled control guide RNA) to specifically knock down Avp in the MePD of WT mice exposed to CSIS (FIG. 5G), which we confirmed with RT-qPCR (FIG. 5H).

Diminished Avp in the MePD decreased marble burying (FIG. 5I) and increased the time spent in the open arms of the EPM (FIG. 5J) in females but not males. Avp knockdown had no effect on time spent in the center of the open field (FIG. 5K) or on locomotor activity (FIG. 5L). Together, these data demonstrate that AVP in the MePD is required for the sex-specific effect of CSIS on anxiety-related behaviors.

### Example 8

### ERα in AVP^{MePD} Neurons Contributes to Sex-Specific Effects of CSIS on Anxiety-Related Behaviors.

The MePD is a sexually dimorphic brain region that regulates sex-specific behaviors, in part through ERα signaling (Chen et al., 2019; Spiteri et al., 2010). Since ERα is co-expressed with AVP in the rat MePD (Axelson and Leeuwen, 1990), we investigated whether it contributes to the effects of CSIS on anxiety-related behaviors in females.

We confirmed that >90% of AVP^{MePD} neurons co-expressed ERα by immunohistochemistry in the Avp-Flp::GFP reporter line (FIG. 15). We used an intersectional approach to exclusively delete Esr1, the gene encoding Era, from AVP^{MePD} neurons, which represent a very small subset of all ERα-positive neurons in the MePD. We generated Avp-Flp::Es1^{flox/flox} mice and injected them with AAV-fDIO-Cre in the MePD (FIG. 5M). We confirmed the specific deletion of Esr1 in AVP^{MePD} neurons by smFISH (FIG. 5N). Deletion of Esr1 from the AVP^{MePD} neurons in the context of CSIS decreased marble burying (FIG. 5O) and increased the time spent in the open arms of the EPM, although it did not reach significance (FIG. 5P), in females but not males. It had no effect on time spent in the center of the open field (FIG. 5Q) or on locomotor activity (FIG. 5R). These data support the idea that estrogen signaling through Era in AVP^{MePD} neurons mediates some of the effects of CSIS on anxiety-related behaviors.

### Methods

### Animals

All animals were maintained on a 12 h/12 h light/dark cycle (7 am lights on), with ad libitum access to food and water, unless stated otherwise. C57BL/6J mice (Jax strain #000664, WT) were used for transcriptomic analyses, behavioral experiments, smFISH, CRISPR knock-down, and pharmacological studies. The Avpr1a-Cre line was generated by the Molecular Genetics Core at the University of Michigan by inserting the P2A-Cre transgene in frame with Avpr1a using CRISPR-mediated gene editing techniques and was used for chemogenetic and tracing studies. The Avpr1a-Cre mouse line was crossed onto the 6.Cg-Gt(ROSA)26Sortm9(CAG-tdTomato)Hze/J reporter line (Ai9, Jax strain #007909). Avpr1a^{Flox} and Avp-Flp lines were generated by Cyagen Biosciences (Santa Clara, CA) and provided by the Dölen laboratory. Cre-dependent Avpr1a knockout mice (Avpr1a^{Flox}) were generated by inserting LoxP sites flanking exon 1 of the mAvpr1a gene.

The targeting vector was generated by PCR using BAC clones RP24-352P7 and RP24-268P17 from the C57BL/6J library as template. Avp-Flp mice were generated by replacing the stop codon in exon 3 of the endogenous mAvp gene with a 2A-Flp construct. The Avp-Flp line was crossed onto the Gt(ROSA)26Sortm1.2(CAG-EGFP)Fsh/Mmjax mouse line (Jax strain #32038), and onto the B6(Cg)-Esr1tm4.1Ksk/J mouse line (Jax strain #032173). All procedures were performed within the guidelines of the Institutional Animal Care and Use Committee (IACUC) at the Columbia University Health Science Division.

### Stresses

Repeated restraint: Animals were restrained in well-ventilated 50 ml tubes and left undisturbed for 1 h on 5 consecutive days.

Overcrowding: Mice were either housed in cages of 4 (control group) or in cages of 8 (overcrowded group) for 7 weeks.

Chronic social Isolation: Mice were singly housed at 5 weeks of age for 7 weeks in standard cages. Control groups included mice isolated at 8 weeks of age for 2 weeks, or at 8 weeks of age for 7 weeks. An additional control group included mice socially isolated at 8 weeks of age for 7 weeks and regrouped at 15 weeks for 3 weeks.

mRNA Extraction

Mice were anesthetized after 7 pm (Avertin, i.p., 0.32 ml/10 g of 2.5% solution, Sigma Aldrich; or isoflurane 5% isoflurane/1 L O2/min) and euthanized by decapitation. For whole amygdala samples, brains were micro-dissected at bregma coordinates −0.58 mm to −2.7 mm. Sub-regions of the amygdala were micro-dissected from two 0.5 mm slices of the brains at bregma −1.0 mm and −2.0 mm with the EMS-Core Sampling Tool (EMS): one punch of 0.35 mm diameter for MeV and BLA; two punches of 0.5 mm diameter for the CeA/MeD; and one punch of 1.0 mm diameter for BMA (FIG. 6). Snap-frozen tissues were homogenized, and mRNA was extracted using the RNeasy Micro Kit (Qiagen).

### RNA Sequencing

High-throughput RNA-sequencing profiles of the whole amygdala were generated in group housed female WT mice during mid-adolescence (5 weeks), late-adolescence (7 weeks), young adulthood (13 weeks) and mature adulthood (22 weeks) from 3 biological replicates of n=3-4 samples. RNA purity was confirmed using a Bioanalyzer (DE72901373) (n=11-12). 3-4 samples of each group were pooled for RNA-seq. Library construction was performed using the Illumina TruSeq Stranded mRNA library prep kit followed by poly-A pull-down. Sequencing was performed on an Illumina NovaSeq 6000 in a multiplex setting (40M paired end, reads 2×100 bp) at the JP Sulzberger Columbia Genome Center Core. Illumina RTA was used for base calling and bcl2fastq2 (version 2.20) was used for converting BCL to fastq format, coupled with adaptor trimming. Illumina FASTQ files were pseudo-aligned to the mouse genome (GRCm38) using kallisto v 0.44.0 (Bray et al., 2016). Transcript abundance estimates were converted into count data using the tximport v 1.6.0 (Soneson et al., 2015) and Ensembl based annotation package Ensembl Db.Mmusculus.v79.

### RNA-Seq Data Analysis

Differential Expression (DE) Analysis: DE was performed with the Bioconductor DESeq2 package (v1.18.1) that uses negative binomial generalized linear models, where the estimates of dispersion and logarithmic fold changes incorporate data-driven prior distributions (Love et al., 2014). Benjamini and Hochberg's algorithm was used to control the false discovery rate (FDR) due to multiple testing (Benjamini and Hochberg, 1995); genes with FDR (q-value)<0.05 were considered differentially expressed. Wald's test was used to test the DE between two-time points with the null hypothesis of no difference. Genes with positive log 2 fold change from weeks 5 to 7 (upregulation) and negative log 2 fold change (downregulation) from weeks 7 to 13 and weeks 13 to 22 were reviewed. The top 300 genes sorted by ascending P values were selected for further analyses.

Gene Ontology Analysis: The top 300 genes with a peak of expression at 7 weeks were classified by molecular function using the web-based PANTHER software (http://www.pantherdb.org/). GeneCards (https://www.genecards.org/) and AmiGO (http://amigo.geneontology.org/amigo) were used to enhance the accuracy of the gene annotations.

Network Analysis: A network analysis was performed using the STRING web software (https://string-db.org/) on the 15 genes with DE at 7 weeks that were classified in the molecular transducer family.

Pathway Analysis: A pathway analysis was performed on the same genes as the network analysis using KEGG pathway mapping web software (https://www.genome.jp/kegg/mapper.html).

qPCR

cDNA was generated from 50-200 ng of total RNA by reverse transcription using the SuperScript™ IV VILO™ Master Mix (Invitrogen). RT-qPCR was performed with the QuantStudio 5 RT-qPCR system, Design & Analysis software, and TaqMan Fast Advanced master mix (Applied Biosystems). Glyceraldehyde-3-phosphate dehydrogenase (Gapdh) was used as housekeeping gene control for normalization of gene expression. TaqMan assays (Applied Biosystems) included: Gapdh, Mm00434129_m1; Crhr2, Mm00438308_m1; Htr4, Mm00434129_m1; Avpr1a, Mm00444092_m1; Gng12, Mm01183812_m1; Slpr3, Mm02620181_s1; Sctr, Mm01290788_m1; Avp, Mm01271704_m1. Relative quantification of gene expression was calculated using the 2−ΔΔCt formula (Schmittgen T D, and Livak K J. Analyzing real-time PCR data by the comparative C (T) method. Nat Protoc. 2008; 3 (6): 1101-8).

Single Molecule Fluorescent In Situ Hybridization (smFISH)

Mice were anesthetized (Avertin, i.p., 0.32 ml/10 g of 2.5% solution) and decapitated. Brains were snap frozen and cut in coronal cryosections (20 μm) and thaw-mounted onto Superfrost Plus® slides (Fisherbrand) prior to storage at −80° C. smFISH was performed using RNAscope® Fluorescent Multiplex Kit (ACDBio). Probes used included: Avpr1a (#418061), Solute carrier family 32 (Slc32a1, #319198), iCre (#423321), Avp (#401391), Gfp (#409018), Esr1 (#49622). Images were taken using the Zeiss LSM 710 confocal microscope (Zeiss). Cell counts were performed manually with Photoshop software.

### Perfusion and Immunohistochemistry

Mice were deeply anesthetized (Avertin, i.p., 0.32 ml/10 g of 2.5% solution) and transcardially perfused with iced-cold physiological saline followed by 4% paraformaldehyde. Mice were decapitated and brains were extracted and post-fixed in 4% paraformaldehyde overnight at 4° C. Brains were then transferred in cryoprotecting 30% sucrose before cryosectioning into four representative series of 30 μm sections and processed for free-floating immunohistochemistry. Primary antibodies used were rabbit anti-DsRed (1:500; #632496, Takara), rat anti-mCherry (1:1000, #16D7, Invitrogen), goat anti-cFos (1:1000, #PA1-18329, Invitrogen), and sheep anti-GFP (1:1000; #4745-1051, Biorad). Secondary antibodies used were donkey anti-rabbit (#A-31572, Invitrogen), donkey anti-rat IgG-Alexa594 (#A-11007, Invitrogen), donkey anti-goat IgG-Alexa488 (#A32814, Invitrogen), donkey anti-sheep IgG-Alexa488 (#A-11015, Invitrogen). Free-floating sections were mounted on microscope slides and immunohistochemistry staining was visualized with the Zeiss LSM 710 confocal microscope (Zeiss).

### Stereotaxic Surgery

Mice were anesthetized with isoflurane (1-5% isoflurane/1 L O2/min) and placed on a double-armed stereotaxic frame (Stoelting). For acute viral injections, ophthalmic ointment and analgesics were administered (buprenorphine, 0.1 mg/kg or buprenorphine Ethiqa XR, 3.25 mg/kg, subcutaneous). A craniotomy was made to insert a guide cannula (Model C315G/SPC, Plastics One) to the CeA (1.34 mm posterior, +/−2.4 mm lateral and 4.5 mm ventral to Bregma according to the Paxinos and Franklin Mouse Brain Atlas (Paxinos and Franklin, 2001)) or to the CPu (1.34 mm posterior, +/−2.95 mm lateral and 3.7 mm ventral to Bregma).

The following viruses were injected::AAV5-hSyn-DIO-hM3Dq-mCherry (DREADD-Gq, titer 6×10¹² cfu/ml, 250 ul bilateral, #44361, Addgene), AAV5-hSyn-DIO-mCherry (control, titer 6×10¹² cfu/ml, 250 nl bilateral, #50459, Addgene), AAV5-hSyn-GFP-Cre (titer 3.5×10¹² cfu/ml, 250 nl bilateral, #6446C, UNC vector core), AAV5-hSyn-EGFP (titer 4×10¹² cfu/ml, 250 nl bilateral, #4657D, UNC vector core), AAV8.2-hEF1a-DIO-Synaptophysin-mCherry (titer 2.5×10¹³ vg/ml, 100 nl unilateral, #AAV-RN1, MGH), AAVrg-hSyn-DIO-EGFP (1.8×10¹³ vg/ml, 100 nl unilateral, #50457, Addgene), AAVrg-Ef1a-fDIO-mCherry (2.2×10¹³ vg/ml, 100 nl unilateral, #114471, Addgene), AAV9-EF1a-fDIO-Cre (1.3×10¹³ vg/ml, 100 nl unilateral, #121675, Addgene), AAV5-EF1a-fDIO-mCherry (1×10¹³ vg/ml, 100 nl unilateral, #121675, Addgene), AAVrg-EF1a-DIO-FLPo-WPRE-hGHpA (titer 1.6×10¹³ vg/ml, 250 nl bilateral, #87306, Addgene), AAV-DJ-hSyn-fDIO-hMD4Gi-mCherry (titer 2×10¹³ vg/ml, 250 nl bilateral, #GVVC-AAV-154, Stanford university gene vector and virus core), pAAV (Exp)-CMV-SaCas9 (titer >2×10¹³ vg/ml, 250 ul bilateral, #AAV9SP(VB210611-1334ntv), VectorBuilder), pAAV2gRNA-EGFP (mouse Avp_gRNA #1) (titer >2×10¹³ vg/ml, 250 ul bilateral, #AAV9SP(VB210611-1330hbv), VectorBuilder), pAAV (2gRNA)-EGFP (scramble) (titer >2×10¹³ vg/ml, 250 ul bilateral, #AAV9SP(VB210615-1067hym), VectorBuilder).

Injections were performed with a unilateral injector (Model C315I/SPC, Plastics One) attached to a Hamilton syringe (0.5 ml; Hamilton Company, Reno, NV) and an infusion pump (Kd Scientific #100) at an infusion rate of 50 nL/min. The cannula and injector remained in place for 5 min to prevent backflow, skull access was then sealed with bone wax (#DYNJBW25, Medline), and the incision was closed with wound clips (#RF7, Braintree scientific).

For cannulations, ophthalmic ointment and analgesics were administered (buprenorphine, 0.1 mg/kg or buprenorphine Ethiqa XR, 3.25 mg/kg, and carprofen, 5 mg/kg, subcutaneous). Two cannulas (Model C317GS, Plastics One) were placed by drilling holes at the following coordinates: +/−2.4 mm lateral, −1.4 mm anteroposterior and −4.6 mm ventral from Bregma. Implants were secured by dental cement and protected with a cap (Model C317DCS, Plastics One).

### Chemogenetic Experiments

To stimulate AVPR1A^{CeA} neurons or to inhibit Avpr1a-expressing neuronal projections from the CeA to the CPu, mice were injected with clozapine-N-oxide (CNO, 1.5 mg/kg, i.p., #SML2304, Sigma-Aldrich) 30 min prior to behavioral testing.

### Behavioral Assays

All behavioral assays were performed after lights out (7 pm).

Marble burying: Standard cages were filled with fresh bedding to a depth of 2.5 in and 15 marbles were evenly spaced across the bedding. Animals were placed in the cage for 30 min and allowed to ambulate freely. At the end of the assay, the number of marbles buried was estimated visually. A marble was considered buried if at least ¾ of its surface was covered by the bedding.

Elevated plus-maze (EPM): The apparatus consisted of a platform elevated 30 cm above the floor with four perpendicular arms: two arms were enclosed by 20 cm high walls and two arms were open. At the beginning of the test, mice were placed into the center zone facing the open arms and allowed to move freely for 15 min while the software recorder the bean breaks in each arm.

Open field: Testing occurred in a Plexiglas box with dimensions of 30 cm width×30 cm length×30 cm height. At the beginning of the test, mice were placed into the corner of the open field box and allowed to move freely for 30 min.

Social recognition: Singly housed WT females were housed in their home cages. On day one, a novel female mouse was introduced into the cage, and behaviors were recorded using ANY-maze software. Time spent sniffing the novel individual was scored manually. On day 2, the novel individual was re-introduced to the same cage, and time spent sniffing was recorded and scored manually.

### Pharmacological Treatments

Intra-amygdala AVP injections: Prior to marble burying, adult WT and Avpr1a^{-/-} females were bilaterally injected with 1 ng of AVP or 0.9% saline via in-dwelling cannulas using a Hamilton syringe (Hamilton 7635-01) connected to an injector (Model C317IS, Plastics One).

Peripheral antagonist injections: Injections of SRX246 (AVPR1A antagonist, 2 mg/kg, i.p.), SR49059 (AVPR1A antagonist, 2 mg/kg, i.p.), SSR149415 (AVPR1B antagonist, 2 mg/kg, i.p.), d(CH2)5Tyr(Me)-[Orn8]-vasotocin (Oxytocin receptor antagonist, 2 mg/kg, i.p.), or 1% DMSO (Sigma Aldrich, i.p.) were performed in WT adults.

### Water Intake Measurements

Water intake was monitored continuously using the BioDAQ automated system (Research Diets). Mice were habituated for 3-4 days to the BioDaq and 4-5 days to a 7 pm-7 am access feeding schedule. Injections were performed at 7 pm. Water intake was analyzed with the BioDaq software.

### Statistics and Reproducibility

All behavioral data was scored by a trained observer blind to experimental conditions, or scored using an automated system (Ethovision, Med Associates). Data were then processed and analyzed using GraphPad Prism 8. First, we performed a Grubbs' test on every dataset in order to exclude any significant outliers. When appropriate, we performed a Shapiro-Wilk test to assess the normality of the distribution of the samples. Statistical analyses were then conducted using two ways RM ANOVAs followed by Tukey or Bonferroni post hoc tests, one way ANOVA followed by Tukey post hoc test, Kruskal-Wallis test followed by Dunn's post hoc test, Mixed-effects analysis followed by Bonferroni post hoc test, and unpaired t-tests when appropriate. Effectives were reported in the figure legends. Statistically significant effects were reported in each figure. The significance threshold was held at α=0.05, two-tailed (not significant, ns, p>0.05; * p<0.05; ** p<0.01; *** p<0.001; **** p<0.0001).

### HIGHLIGHTS AND CONCLUSIONS

Any previous assertions that AVPR1A antagonists can be used to treat “anxiety” in males and females were based on naïve assumptions about the generalizability of early findings in males tested in the context of the resident-intruder model of offensive aggression (Ferris, Lu et al. 2006, Ferris 2008). It is now well established that effects of the AVP system are highly dependent on context, as opposite effects can be elicited, depending on the sex, brain region and type of stressor under investigation.

For the first time, we identified AVPR1A as a therapeutic target for anxiety caused by social isolation in females. This identification is specific to sex (female), brain region (central nucleus of the amygdala (CeA)) and stressor (social isolation) that have never been evaluated by previous claimants.

Social isolation leads to an upregulation of AVPR1A in the central nucleus of the amygdala (CeA) in females and not males. This effect is relatively specific to the CeA.

Disruption of signaling from AVPR1A in the CeA by chemogenetic or genetic means markedly reduces anxiety- and OCD-like behavior associated with social isolation in females. Anxiolytic effects of the AVPR1A antagonist SRX246 are specific to socially-isolated females, as they are not seen in males or group-housed females. (The antagonist may even slightly increase anxiety in males, which would be consistent with findings that AVP inhibited hyper aggression induced by social isolation in male mice (Tan, Musullulu et al. 2019)). Studies are underway to evaluate the efficacy of an antisense oligo in both females and males.

### REFERENCES

Albers, H. E., Pollock, J., Simmons, W. H., and Ferris, C. F. (1986). A V1-like receptor mediates vasopressin-induced flank marking behavior in hamster hypothalamus. J Neurosci 6, 2085-2089.
Arakawa, H. (2018). Ethological approach to social isolation effects in behavioral studies of laboratory rodents. Behav Brain Res 341, 98-108.
Arakawa, H., Arakawa, K., and Deak, T. (2010). Oxytocin and vasopressin in the medial amygdala differentially modulate approach and avoidance behavior toward illness-related social odor. Neuroscience 171, 1141-1151.
Axelson, J. F., and Leeuwen, F. W. (1990). Differential localization of estrogen receptors in various vasopressin synthesizing nuclei of the rat brain. J Neuroendocrinol 2, 209-216.
Azzopardi, E., Louttit, A. G., DeOliveira, C., Laviolette, S. R., and Schmid, S. (2018). The Role of Cholinergic Midbrain Neurons in Startle and Prepulse Inhibition. J Neurosci 38, 8798-8808.
Bale, T. L., and Epperson, C. N. (2015). Sex differences and stress across the lifespan. Nat Neurosci 18, 1413-1420.
Barchiesi, R., Chanthongdee, K., Domi, E., Gobbo, F., Coppola, A., Asratian, A., Toivainen, S., Holm, L., Augier, G., Xu, L., et al. (2021). Stress-induced escalation of alcohol self-administration, anxiety-like behavior, and elevated amygdala Avp expression in a susceptible subpopulation of rats. Addict Biol 26, e13009.
Baxter, A. J., Scott, K. M., Vos, T., and Whiteford, H. A. (2013). Global prevalence of anxiety disorders: a systematic review and meta-regression. Psychol Med 43, 897-910.
Bayerl, D. S., Honig, J. N., and Bosch, O. J. (2016). Vasopressin Vla, but not V1b, receptors within the PVN of lactating rats mediate maternal care and anxiety-related behaviour. Behav Brain Res 305, 18-22.
Beck, K. D., and Luine, V. N. (2002). Sex differences in behavioral and neurochemical profiles after chronic stress: role of housing conditions. Physiol Behav 75, 661-673.
Beiderbeck, D. I., Neumann, I. D., and Veenema, A. H. (2007). Differences in intermale aggression are accompanied by opposite vasopressin release patterns within the septum in rats bred for low and high anxiety. Eur J Neurosci 26, 3597-3605.
Benenson, J. F., Markovits, H., Hultgren, B., Nguyen, T., Bullock, G., and Wrangham, R. (2013). Social exclusion: more important to human females than males. PloS one 8, e55851.
Benjamini, Y., and Hochberg, Y. (1995). Controlling the False Discovery Rate: A Practical and Powerful Approach to Multiple Testing. J of the Royal Statistical Society, Series B (Methodological) 57, 289-300.
Bielsky, I. F., Hu, S. B., Ren, X., Terwilliger, E. F., and Young, L. J. (2005a). The Vla vasopressin receptor is necessary and sufficient for normal social recognition: a gene replacement study. Neuron 47, 503-513.
Bielsky, I. F., Hu, S. B., Szegda, K. L., Westphal, H., and Young, L. J. (2004). Profound impairment in social recognition and reduction in anxiety-like behavior in vasopressin Vla receptor knockout mice. Neuropsychopharmacology 29, 483-493.
Bielsky, I. F., Hu, S. B., and Young, L. J. (2005b). Sexual dimorphism in the vasopressin system: lack of an altered behavioral phenotype in female Vla receptor knockout mice. Behav Brain Res 164, 132-136.
Bluthe, R. M., and Dantzer, R. (1990). Social recognition does not involve vasopressinergic neurotransmission in female rats. Brain Res 535, 301-304.
Bourhy, L., Mazeraud, A., Costa, L. H. A., Levy, J., Rei, D., Hecquet, E., Gabanyi, I., Bozza, F. A., Chretien, F., Lledo, P. M., et al. (2022). Silencing of amygdala circuits during sepsis prevents the development of anxiety-related behaviours. Brain 145, 1391-1409.
Bray, N. L., Pimentel, H., Melsted, P., and Pachter, L. (2016). Near-optimal probabilistic RNA-seq quantification. Nat Biotechnol 34, 525-527.
Bredewold, R., Smith, C. J., Dumais, K. M., and Veenema, A. H. (2014). Sex-specific modulation of juvenile social play behavior by vasopressin and oxytocin depends on social context. Front Behav Neurosci 8, 216.
Bredewold, R., and Veenema, A. H. (2018). Sex differences in the regulation of social and anxiety-related behaviors: insights from vasopressin and oxytocin brain systems. Curr Opin Neurobiol 49, 132-140.
Broekkamp, C. L., Rijk, H. W., Joly-Gelouin, D., and Lloyd, K. L. (1986). Major tranquillizers can be distinguished from minor tranquillizers on the basis of effects on marble burying and swim-induced grooming in mice. Eur J Pharmacol 126, 223-229.
Brot, M. D., De Vries, G. J., and Dorsa, D. M. (1993). Local implants of testosterone metabolites regulate vasopressin mRNA in sexually dimorphic nuclei of the rat brain. Peptides 14, 933-940.
Brown, G. W. (1993). The role of life events in the aetiology of depressive and anxiety disorders. In Stress: from synapse to syndrome, S. P. Stannford S, ed. (London: Academic Press), pp. 23-50.
Brownstein, M. J., Simon, N. G., Long, J. D., Yankey, J., Maibach, H. T., Cudkowicz, M., Coffey, C., Conwit, R. A., Lungu, C., Anderson, K. E., et al. (2020). Safety and Tolerability of SRX246, a Vasopressin 1a Antagonist, in Irritable Huntington's Disease Patients-A Randomized Phase 2 Clinical Trial. J Clin Med 9.
Cacioppo, J. T., Cacioppo, S., Cole, S. W., Capitanio, J. P., Goossens, L., and Boomsma, D. I. (2015). Loneliness across phylogeny and a call for comparative studies and animal models. Perspect Psychol Sci 10, 202-212.
Chen, P. B., Hu, R. K., Wu, Y. E., Pan, L., Huang, S., Micevych, P. E., and Hong, W. (2019). Sexually Dimorphic Control of Parenting Behavior by the Medial Amygdala. Cell 176, 1206-1221 e1218.
Clauss, N., and Byrd-Craven, J. (2019). Exposure to a sex-specific stressor mitigates sex differences in stress-induced eating. Physiol Behav 202, 26-35.
Coccaro, E. F., Kavoussi, R. J., Hauger, R. L., Cooper, T. B., and Ferris, C. F. (1998). Cerebrospinal fluid vasopressin levels: correlates with aggression and serotonin function in personality-disordered subjects. Arch Gen Psychiatry 55, 708-714.
Collaborators, C.-M. D. (2021). Global prevalence and burden of depressive and anxiety disorders in 204 countries and territories in 2020 due to the COVID-19 pandemic. Lancet 398, 1700-1712.
Craske, M. G. (2003). Origins of phobias and anxiety disorders: why more women than men? (Amsterdam: Elsevier).
Dantzer, R., Koob, G. F., Bluthe, R. M., and Le Moal, M. (1988). Septal vasopressin modulates social memory in male rats. Brain Res 457, 143-147.
De Vries, G. J., al-Shamma, H. A., and Zhou, L. (1994a). The sexually dimorphic vasopressin innervation of the brain as a model for steroid modulation of neuropeptide transmission. Ann N Y Acad Sci 743, 95-120.
De Vries, G. J., Wang, Z., Bullock, N. A., and Numan, S. (1994b). Sex differences in the effects of testosterone and its metabolites on vasopressin messenger RNA levels in the bed nucleus of the stria terminalis of rats. J Neurosci 14, 1789-1794.
DeVries, G. J., Buijs, R. M., Van Leeuwen, F. W., Caffe, A. R., and Swaab, D. F. (1985). The vasopressinergic innervation of the brain in normal and castrated rats. J Comp Neurol 233, 236-254.
Donner, N. C., and Lowry, C. A. (2013). Sex differences in anxiety and emotional behavior. Pflugers Arch 465, 601-626.
Everts, H. G., and Koolhaas, J. M. (1999). Differential modulation of lateral septal vasopressin receptor blockade in spatial learning, social recognition, and anxiety-related behaviors in rats. Behav Brain Res 99, 7-16.
Fabio, K., Guillon, C., Lacey, C. J., Lu, S. F., Heindel, N. D., Ferris, C. F., Placzek, M., Jones, G., Brownstein, M. J., and Simon, N. G. (2012). Synthesis and evaluation of potent and selective human Vla receptor antagonists as potential ligands for PET or SPECT imaging. Bioorganic & medicinal chemistry 20, 1337-1345.
Francois, M., Canal Delgado, I., Shargorodsky, N., Leu, C. S., and Zeltser, L. (2022). Assessing the effects of stress on feeding behaviors in laboratory mice. eLife 11.
Francois, M., Fernandez-Gayol, O., and Zeltser, L. M. (2021). A Framework for Developing Translationally Relevant Animal Models of Stress-Induced Changes in Eating Behavior. Biol Psychiatry.
Furman, O., Tsoory, M., and Chen, A. (2022). Differential chronic social stress models in male and female mice. Eur J Neurosci 55, 2777-2793.
Gillies, G. E., Linton, E. A., and Lowry, P. J. (1982). Corticotropin releasing activity of the new CRF is potentiated several times by vasopressin. Nature 299, 355-357.
Goel, N., and Bale, T. L. (2009). Examining the intersection of sex and stress in modelling neuropsychiatric disorders. J Neuroendocrinol 21, 415-420.
Griebel, G., Stemmelin, J., Gal, C. S., and Soubrie, P. (2005). Non-peptide vasopressin V1b receptor antagonists as potential drugs for the treatment of stress-related disorders. Curr Pharm Des 11, 1549-1559.
Haller, J., Fuchs, E., Halasz, J., and Makara, G. B. (1999). Defeat is a major stressor in males while social instability is stressful mainly in females: towards the development of a social stress model in female rats. Brain Res Bull 50, 33-39.
Heinrichs, M., and Domes, G. (2008). Neuropeptides and social behaviour: effects of oxytocin and vasopressin in humans. Prog Brain Res 170, 337-350.
Hernandez-Perez, O. R., Crespo-Ramirez, M., Cuza-Ferrer, Y., Anias-Calderon, J., Zhang, L., Roldan-Roldan, G., Aguilar-Roblero, R., Borroto-Escuela, D. O., Fuxe, K., and Perez de la Mora, M. (2018). Differential activation of arginine-vasopressin receptor subtypes in the amygdaloid modulation of anxiety in the rat by arginine-vasopressin. Psychopharmacology (Berl) 235, 1015-1027.
Hernandez, V. S., Hernandez, O. R., Perez de la Mora, M., Gomora, M. J., Fuxe, K., Eiden, L. E., and Zhang, L. (2016). Hypothalamic Vasopressinergic Projections Innervate Central Amygdala GABAergic Neurons: Implications for Anxiety and Stress Coping. Front Neural Circuits 10, 92.
Herrero, M. J., Velmeshev, D., Hernandez-Pineda, D., Sethi, S., Sorrells, S., Banerjee, P., Sullivan, C., Gupta, A. R., Kriegstein, A. R., and Corbin, J. G. (2020). Identification of amygdala-expressed genes associated with autism spectrum disorder. Molecular autism 11, 39.
Hilakivi, L. A., Ota, M., and Lister, R. G. (1989). Effect of isolation on brain monoamines and the behavior of mice in tests of exploration, locomotion, anxiety and behavioral ‘despair’. Pharmacol Biochem Behav 33, 371-374.
Hodes, G. E., and Epperson, C. N. (2019). Sex Differences in Vulnerability and Resilience to Stress Across the Life Span. Biol Psychiatry 86, 421-432.
House, J. S., Landis, K. R., and Umberson, D. (1988). Social relationships and health. Science 241, 540-545.
Kessler, R. C., Berglund, P., Demler, O., Jin, R., Merikangas, K. R., and Walters, E. E. (2005). Lifetime prevalence and age-of-onset distributions of DSM-IV disorders in the National Comorbidity Survey Replication. Arch Gen Psychiatry 62, 593-602.
Kessler, R. C., Petukhova, M., Sampson, N. A., Zaslavsky, A. M., and Wittchen, H. U. (2012). Twelve-month and lifetime prevalence and lifetime morbid risk of anxiety and mood disorders in the United States. Int J Methods Psychiatr Res 21, 169-184.
Kim, J., Zhang, X., Muralidhar, S., LeBlanc, S. A., and Tonegawa, S. (2017). Basolateral to Central Amygdala Neural Circuits for Appetitive Behaviors. Neuron 93, 1464-1479 e1465.
Klaser, K., Thompson, E. J., Nguyen, L. H., Sudre, C. H., Antonelli, M., Murray, B., Canas, L. S., Molteni, E., Graham, M. S., Kerfoot, E., et al. (2021). Anxiety and depression symptoms after COVID-19 infection: results from the COVID Symptom Study app. J Neurol Neurosurg Psychiatry 92, 1254-1258.
Knowlton, B. J., Mangels, J. A., and Squire, L. R. (1996). A neostriatal habit learning system in humans. Science 273, 1399-1402.
Lago, T., Davis, A., Grillon, C., and Ernst, M. (2017). Striatum on the anxiety map: Small detours into adolescence. Brain Res 1654, 177-184.
Landgraf, R. (2006). The involvement of the vasopressin system in stress-related disorders. CNS Neurol Disord Drug Targets 5, 167-179.
Landgraf, R., and Neumann, I. D. (2004). Vasopressin and oxytocin release within the brain: a dynamic concept of multiple and variable modes of neuropeptide communication. Front Neuroendocrinol 25, 150-176.
Langer, E., Einat, H., and Stukalin, Y. (2020). Similarities and dissimilarities in the effects of benzodiazepines and specific serotonin reuptake inhibitors (SSRIs) in the defensive marble burying test: A systematic review and meta-analysis. Eur Neuropsychopharmacol 36, 38-49.
Lingawi, N. W., and Balleine, B. W. (2012). Amygdala central nucleus interacts with dorsolateral striatum to regulate the acquisition of habits. J Neurosci 32, 1073-1081.
Liu, X., Wu, R., Tai, F., Ma, L., Wei, B., Yang, X., Zhang, X., and Jia, R. (2013). Effects of group housing on stress induced emotional and neuroendocrine alterations. Brain Res 1502, 71-80.
Love, M. I., Huber, W., and Anders, S. (2014). Moderated estimation of fold change and dispersion for RNA-seq data with DESeq2. Genome Biol 15, 550.
Luciano, D., and Lore, R. (1975). Aggression and social experience in domesticated rats. J Comp Physiol Psychol 88, 917-923.
Manning, C. J., Dewsbury, D. A., Wakeland, E. K., and Potts, W. K. (1995). Communal nesting and communal nursing in house mice, Mus musculus domesticus. Anim Behav 50, 741-751.
McEwen, B. S., and Stellar, E. (1993). Stress and the individual. Mechanisms leading to disease. Archives of internal medicine 153, 2093-2101.
Meyer-Lindenberg, A., Domes, G., Kirsch, P., and Heinrichs, M. (2011). Oxytocin and vasopressin in the human brain: social neuropeptides for translational medicine. Nat Rev Neurosci 12, 524-538.
Montgomery, K. C. (1958). The relation between fear induced by novel stimulation and exploratory behavior. J Comp Physiol Psychol 48, 254-260.
Murgatroyd, C., Wigger, A., Frank, E., Singewald, N., Bunck, M., Holsboer, F., Landgraf, R., and Spengler, D. (2004). Impaired repression at a vasopressin promoter polymorphism underlies overexpression of vasopressin in a rat model of trait anxiety. J Neurosci 24, 7762-7770.
Neumann, I. D., and Landgraf, R. (2012). Balance of brain oxytocin and vasopressin: implications for anxiety, depression, and social behaviors. Trends Neurosci 35, 649-659.
Oliveira, V. E. M., Neumann, I. D., and de Jong, T. R. (2019). Post-weaning social isolation exacerbates aggression in both sexes and affects the vasopressin and oxytocin system in a sex-specific manner. Neuropharmacology 156, 107504.
Ordaz, S., and Luna, B. (2012). Sex differences in physiological reactivity to acute psychosocial stress in adolescence. Psychoneuroendocrinology 37, 1135-1157.
Palanza, P. (2001). Animal models of anxiety and depression: how are females different? Neurosci Biobehav Rev 25, 219-233.
Patriquin, M. A., and Mathew, S. (2017). The Neurobiological Mechanisms of Generalized Anxiety Disorder and Chronic Stress. Chronic Stress (Thousand Oaks) 1.
Paxinos, G., and Franklin, K. B. J. (2001). The Mouse Brain in Stereotaxic Coordinates, 2nd edn (San Diego, Calif; London: Academic Press).
Pine, D. S., Cohen, P., Gurley, D., Brook, J., and Ma, Y. (1998). The risk for early-adulthood anxiety and depressive disorders in adolescents with anxiety and depressive disorders. Arch Gen Psychiatry 55, 56-64.
Plumari, L., Viglietti-Panzica, C., Allieri, F., Honda, S., Harada, N., Absil, P., Balthazart, J., and Panzica, G. C. (2002). Changes in the arginine-vasopressin immunoreactive systems in male mice lacking a functional aromatase gene. J Neuroendocrinol 14, 971-978.
Rivera-Irizarry, J. K., Skelly, M. J., and Pleil, K. E. (2020). Social Isolation Stress in Adolescence, but not Adulthood, Produces Hypersocial Behavior in Adult Male and Female C57BL/6J Mice. Front Behav Neurosci 14, 129.
Rosso, M., Wirz, R., Loretan, A. V., Sutter, N. A., Pereira da Cunha, C. T., Jaric, I., Wurbel, H., and Voelkl, B. (2022). Reliability of common mouse behavioural tests of anxiety: A systematic review and meta-analysis on the effects of anxiolytics. Neurosci Biobehav Rev 143, 104928.
Rutter, M., Caspi, A., and Moffitt, T. E. (2003). Using sex differences in psychopathology to study causal mechanisms: unifying issues and research strategies. J Child Psychol Psychiatry 44, 1092-1115.
Scordalakes, E. M., and Rissman, E. F. (2004). Aggression and arginine vasopressin immunoreactivity regulation by androgen receptor and estrogen receptor alpha. Genes Brain Behav 3, 20-26.
Seiler, J. L., Cosme, C. V., Sherathiya, V. N., Schaid, M. D., Bianco, J. M., Bridgemohan, A. S., and Lerner, T. N. (2022). Dopamine signaling in the dorsomedial striatum promotes compulsive behavior. Curr Biol 32, 1175-1188 e1175.
Shapiro, R. A., Xu, C., and Dorsa, D. M. (2000). Differential transcriptional regulation of rat vasopressin gene expression by estrogen receptor alpha and beta. Endocrinology 141, 4056-4064.
Sivukhina, E. V., and Jirikowski, G. F. (2021). Oxytocin, but not arginine-vasopressin neurons project from the hypothalamus to amygdala in human: Dil-based tracing study in postmortem brain. J Chem Neuroanat 111, 101882.
Smith, K. S., and Graybiel, A. M. (2013). A dual operator view of habitual behavior reflecting cortical and striatal dynamics. Neuron 79, 361-374.
Somponpun, S., and Sladek, C. D. (2002). Role of estrogen receptor-beta in regulation of vasopressin and oxytocin release in vitro. Endocrinology 143, 2899-2904.
Soneson, C., Love, M. I., and Robinson, M. D. (2015). Differential analyses for RNA-seq: transcript-level estimates improve gene-level inferences. F1000Res 4, 1521.
Spiteri, T., Musatov, S., Ogawa, S., Ribeiro, A., Pfaff, D. W., and Agmo, A. (2010). The role of the estrogen receptor alpha in the medial amygdala and ventromedial nucleus of the hypothalamus in social recognition, anxiety and aggression. Behav Brain Res 210, 211-220.
Stevens, J. S., and Hamann, S. (2012). Sex differences in brain activation to emotional stimuli: a meta-analysis of neuroimaging studies. Neuropsychologia 50, 1578-1593.
Stroud, L. R., Salovey, P., and Epel, E. S. (2002). Sex differences in stress responses: social rejection versus achievement stress. Biol Psychiatry 52, 318-327.
Szuhany, K. L., and Simon, N. M. (2022). Anxiety Disorders: A Review. Jama 328, 2431-2445.
Takahashi, A., Chung, J. R., Zhang, S., Zhang, H., Grossman, Y., Aleyasin, H., Flanigan, M. E., Pfau, M. L., Menard, C., Dumitriu, D., et al. (2017). Establishment of a repeated social defeat stress model in female mice. Sci Rep 7, 12838.
Tan, T., Wang, W., Liu, T., Zhong, P., Conrow-Graham, M., Tian, X., and Yan, Z. (2021). Neural circuits and activity dynamics underlying sex-specific effects of chronic social isolation stress. Cell Rep 34, 108874.
Taquet, M., Geddes, J. R., Husain, M., Luciano, S., and Harrison, P. J. (2021). 6-month neurological and psychiatric outcomes in 236 379 survivors of COVID-19: a retrospective cohort study using electronic health records. Lancet Psychiatry 8, 416-427.
Taylor, S., Abramowitz, J. S., and Mckay, D. (2012). Non-adherence and non-response in the treatment of anxiety disorders. Journal of anxiety disorders 26, 583-589.
Torruella-Suarez, M. L., Vandenberg, J. R., Cogan, E. S., Tipton, G. J., Teklezghi, A., Dange, K., Patel, G. K., McHenry, J. A., Hardaway, J. A., Kantak, P. A., et al. (2020). Manipulations of Central Amygdala Neurotensin Neurons Alter the Consumption of Ethanol and Sweet Fluids in Mice. J Neurosci 40, 632-647.
Tovote, P., Esposito, M. S., Botta, P., Chaudun, F., Fadok, J. P., Markovic, M., Wolff, S. B., Ramakrishnan, C., Fenno, L., Deisseroth, K., et al. (2016). Midbrain circuits for defensive behaviour. Nature 534, 206-212.
Tye, K. M., Prakash, R., Kim, S. Y., Fenno, L. E., Grosenick, L., Zarabi, H., Thompson, K. R., Gradinaru, V., Ramakrishnan, C., and Deisseroth, K. (2011). Amygdala circuitry mediating reversible and bidirectional control of anxiety. Nature 471, 358-362.
van Leeuwen, F. W., Caffe, A. R., and De Vries, G. J. (1985). Vasopressin cells in the bed nucleus of the stria terminalis of the rat: sex differences and the influence of androgens. Brain Res 325, 391-394.
Veenema, A. H., Blume, A., Niederle, D., Buwalda, B., and Neumann, I. D. (2006). Effects of early life stress on adult male aggression and hypothalamic vasopressin and serotonin. Eur J Neurosci 24, 1711-1720.
Veenema, A. H., Bredewold, R., and De Vries, G. J. (2012). Vasopressin regulates social recognition in juvenile and adult rats of both sexes, but in sex- and age-specific ways. Horm Behav 61, 50-56.
Veenema, A. H., Bredewold, R., and Neumann, I. D. (2007). Opposite effects of maternal separation on intermale and maternal aggression in C57BL/6 mice: link to hypothalamic vasopressin and oxytocin immunoreactivity. Psychoneuroendocrinology 32, 437-450.
Vilhena-Franco, T., Mecawi, A. S., Almeida-Pereira, G., Lucio-Oliveira, F., Elias, L. L. K., and Antunes-Rodrigues, J. (2019). Oestradiol acts through its beta receptor to increase vasopressin neuronal activation and secretion induced by dehydration. J Neuroendocrinol 31, e12712.
Walker, D. M., Cunningham, A. M., Gregory, J. K., and Nestler, E. J. (2019). Long-Term Behavioral Effects of Post-weaning Social Isolation in Males and Females. Front Behav Neurosci 13, 66.
Wang, Y., Krabbe, S., Eddison, M., Henry, F. E., Fleishman, G., Lemire, A. L., Wang, L., Korff, W., Tillberg, P. W., Luthi, A., et al. (2023). Multimodal mapping of cell types and projections in the central nucleus of the amygdala. eLife 12.
Wang, Z. (1994). Testosterone effects on development of vasopressin messenger RNA expression in the bed nucleus of the stria terminalis and medial amygdaloid nucleus in male rats. Brain Res Dev Brain Res 79, 147-150.
Wang, Z., and De Vries, G. J. (1995). Androgen and estrogen effects on vasopressin messenger RNA expression in the medial amygdaloid nucleus in male and female rats. J Neuroendocrinol 7, 827-831.
Weera, M. M., Agoglia, A. E., Douglass, E., Jiang, Z., Rajamanickam, S., Shackett, R. S., Herman, M. A., Justice, N. J., and Gilpin, N. W. (2022). Generation of a CRF (1)-Cre transgenic rat and the role of central amygdala CRF (1) cells in nociception and anxiety-like behavior. eLife 11.
Wittchen, H. U., Nelson, C. B., and Lachner, G. (1998). Prevalence of mental disorders and psychosocial impairments in adolescents and young adults. Psychol Med 28, 109-126.
Yin, H. H., and Knowlton, B. J. (2006). The role of the basal ganglia in habit formation. Nat Rev Neurosci 7, 464-476.
Yorgason, J. T., Espana, R. A., Konstantopoulos, J. K., Weiner, J. L., and Jones, S. R. (2013). Enduring increases in anxiety-like behavior and rapid nucleus accumbens dopamine signaling in socially isolated rats. Eur J Neurosci 37, 1022-1031.
Zelikowsky, M., Hui, M., Karigo, T., Choe, A., Yang, B., Blanco, M. R., Beadle, K., Gradinaru, V., Deverman, B. E., and Anderson, D. J. (2018). The Neuropeptide Tac2 Controls a Distributed Brain State Induced by Chronic Social Isolation Stress. Cell 173, 1265-1279 e1219.

It is to be understood that the agents and methods for treating or preventing social isolation-induced anxiety in females are not limited to the specific embodiments described above, but encompass any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A composition useful for treating anxiety or obsessive-compulsive disorder associated with social isolation in a female subject, wherein the composition comprises a compound that blocks or reduces AVPR1A signaling in the amygdala of the subject.

2. The composition useful for treating anxiety or obsessive-compulsive disorder as set forth in claim 1, wherein the female subject is then-currently experiencing social isolation.

3. The composition useful for treating anxiety or obsessive-compulsive disorder as set forth in claim 1, wherein the compound comprises an AVPR1A antagonist.

4. The composition useful for treating anxiety or obsessive-compulsive disorder as set forth in claim 1, wherein the compound comprises SRX246.

5. The composition useful for treating anxiety or obsessive-compulsive disorder as set forth in any of claims-1-4 claim 1, wherein the compound blocks or reduces AVPR1A signaling in the central nucleus of the amygdala (CeA) of the subject.

6. A method of using a compound that blocks or reduces AVPR1A signaling in a female subject in order to treat or reduce anxiety or obsessive-compulsive disorder associated with social isolation, the method comprising administering to the subject an effective amount of the composition of claim 1.

7. A method of treating or reducing anxiety or obsessive-compulsive disorder associated with social isolation in a female subject, the method comprising administering to the female subject a composition comprising an effective amount of a compound blocking or reducing AVPR1A signaling in the subject.

8. The method of treating or reducing anxiety or obsessive-compulsive disorder as set forth in claim 7, wherein the female subject is then-currently experiencing social isolation.

9. The method of treating or reducing anxiety or obsessive-compulsive disorder as claim 7, wherein the compound comprises an AVPR1A antagonist.

10. The method of treating or reducing anxiety or obsessive-compulsive disorder as set forth in claim 2, wherein the compound blocks or reduces AVPR1A signaling in the central nucleus of the amygdala (CeA) of the subject.

11. The method of treating or reducing anxiety or obsessive-compulsive disorder as set forth in claim 10, wherein the composition comprises SRX246.

12. The composition useful for treating anxiety or obsessive-compulsive disorder as set forth in claim 2, wherein the compound comprises an AVPR1A antagonist.

13. The composition useful for treating anxiety or obsessive-compulsive disorder as set forth in claim 2, wherein the compound comprises SRX246.

14. The composition useful for treating anxiety or obsessive-compulsive disorder as set forth in claim 3, wherein the compound comprises SRX246.

15. The composition useful for treating anxiety or obsessive-compulsive disorder as set forth in claim 2, wherein the compound blocks or reduces AVPR1A signaling in the central nucleus of the amygdala (CeA) of the subject.

16. The composition useful for treating anxiety or obsessive-compulsive disorder as set forth in claim 4, wherein the compound blocks or reduces AVPR1A signaling in the central nucleus of the amygdala (CeA) of the subject.

17. A method of using a compound that blocks or reduces AVPR1A signaling in a female subject in order to treat or reduce anxiety or obsessive-compulsive disorder associated with social isolation, the method comprising administering to the subject an effective amount of the composition of claim 4.

18. The method of treating or reducing anxiety or obsessive-compulsive disorder as set forth in claim 8, wherein the compound comprises an AVPR1A antagonist.

19. The method of treating or reducing anxiety or obsessive-compulsive disorder as set forth in claim 18, wherein the composition comprises SRX246.

20. The method of treating or reducing anxiety or obsessive-compulsive disorder as set forth in claim 7, wherein the composition comprises SRX246.
